(19) United States
(12) Patent Application Publication
(10) Pub. No.: US 2011/0066160 A1
(43) Pub. Date: Mar. 17, 2011

(54) SYSTEMS AND METHODS FOR INSERTING STEERABLE ARRAYS INTO ANATOMICAL STRUCTURES

(75) Inventors: Nabil Simaan, Nashville, TN (US); Jian Zhang, New York, NY (US); Spiros Manolidis, New York, NY (US); J. Thomas Roland, JR., New York, NY (US)

(73) Assignee: THE TRUSTEES OF COLUMBIA UNIVERSITY IN THE CITY OF NEW YORK, New York, NY (US)

(21) Appl. No.: 12/934,569

(22) PCT Filed: Apr. 3, 2009
(86) PCT No.: PCT/US09/39542

§ 371 Date: Nov. 22, 2010

Related U.S. Application Data
(62) Division of application No. 61/042,104, filed on Apr. 3, 2008.
Application No. 61/042,104 is a division of application No. 61/144,018, filed on Jan. 12, 2009.

(51) Int. Cl. A61B 19/00

(52) U.S. Cl. 606/129

(57) ABSTRACT

Systems and Methods are provided for inserting a steerable array into an anatomical structure of the body. The system includes an insertion module for holding a proximal end of the steerable array and a force sensor configured to detect force on the steerable array and to produce force information. The system includes a position sensor configured to detect a position of the insertion module and to produce position information, the position information including a lateral position along an insertion axis and a first approach angle relative to a first reference axis. The system further includes a processor configured to receive the force information from the force sensor and the position information from the position sensor. The processor outputs performance information to a user. The performance information includes an indication of a first differential approach angle relative to an insertion path plan.

### CROSS-REFERENCE TO RELATED APPLICATION

This application claims the benefit of U.S. Provisional Patent Application No. 61/042,104, filed on Apr. 3, 2008, entitled “Robot-Assisted Insertion of Steerable Electrode Arrays,” and U.S. Provisional Patent Application No. 61/144,018, filed on Jan. 12, 2009, entitled “Robotic Tools for Insertion of Cochlear Implant Electrodes,” which are hereby incorporated by reference herein in their entirety.

This invention was made with Government Support under Grant No. 0651649 awarded by the National Science Foundation (CBET). The Government has certain rights in the invention.

### TECHNOLOGY AREA

The disclosed subject matter relates to systems and methods for inserting steerable arrays into anatomical structures.

### BACKGROUND

Cochlear implants have been a major advent in the field of hearing repair. Cochlear implants have aided patients suffering from severe hearing loss due to damaged neuroepithelial cells of the inner ear. Typically, during cochlear implant surgery, a cochlear implant is placed under the skin in a small dimple carved in the mastoid bone. The implant comprises a receiver and a delicate, highly flexible beam called an electrode array that is inserted into the cochlea. The receiver receives (e.g., from an external microphone with a processor and a transmitter) and delivers the necessary excitation to the auditory nerve via the electrode array. In this way, the electrode array restores some sense of hearing by bypassing damaged neuroepithelial cells (hair cells) in the inner ear and directly providing electrical stimulation to the auditory nerve. The cochlear implant system consists of the microphone, micro-processor, transmitter, receiver, and electrode array. FIG. 1, described in detail below, illustrates the cochlear implant system in relation to the inner and outer ear of the patient. The microphone and processor convert sound waves into electrical signals that are wirelessly transmitted to a receiver embedded in the mastoid bone. These electrical signals are then used to excite specific electrodes in the electrode array. These electrodes correspond to different sound frequencies that can be restored by direct excitation of the auditory nerves.

During insertion, the electrode array is usually inserted into the cochlea through a round window into the scala tympani channel. This surgery involves a high level of risk because injuring the basilar membrane can result in complete loss of residual hearing. The anatomic structure of the cochlea and its cross section are shown in FIG. 2A-B. In FIG. 2A, the cochlear implant system is shown in relation to a detailed view of the cochlear anatomy. The device 110 includes a receiver 211, a transmitter 212, and microphone processor 213 positioned in relation to the outer ear and the electrode array 115 positioned within the cochlea, in a selected relation to each portion of the cochlear anatomy. In FIG. 2B, a cross sectional view of the cochlear anatomy is depicted. The cochlear anatomy includes the scala tympani 201, the scala media 202, the scala vestibule 203, the basilar membrane 204, and the auditory nerve 205. The electrode array 115 is positioned to enter the scals tympani. In cochlear implant surgery, the long, thin, and flimsy electrode array 115 is carefully inserted into the scala tympani 201 or scala vestibule 203.

The success and applicability of cochlear implants are currently limited by several factors. For example, during cochlear implantation, electrode array insertion is performed “blindly,” without controlling the interaction of the electrode array and cochlear duct. Also, for example, during implantation, the electrode array can buckle (e.g., from impacting the inner ear) and be rendered nonfunctional. Because of the risk, this surgery is typically performed on a limited subset of the population. Regardless of the approach used, it is evident from previous works that these electrode insertions can easily cause intracochlear trauma. Inventors have identified that this is due to lack of realtime imaging assistance, lack of force feedback during the insertion process, and the lack of controllability of the flexible electrode arrays.

In order to reduce trauma, various electrode arrays have been designed. Flexible and coiled electrode array designs have been proposed by MedEl Corp. (FLEXsoft Electrode), Cochlear Inc. (Contour Advance Electrode), and Advanced Bionics (Hi-Focus Helix Electrode). These electrode arrays are designed to passively bend to accommodate the curvature of the scala tympani during insertions. The present application presents electrode arrays and insertion mechanisms by which intracochlear trauma is significantly reduced.

In U.S. patent application Ser. No. 11/581,899, filed 16 Oct. 2006, entitled “Robot-Assisted Insertion and Monitoring of Passive and Active Bending Cochlear Electrode Arrays,” the entire contents of which are herein incorporated by reference, it was shown that robot-assisted insertion of steerable electrode arrays can have many benefits. In that application, it was shown that an active-bending electrode array can be inserted in the cochlea to restore hearing loss. Force was applied to an actuation thread in the active-bending electrode array to create a deflection in the array. This deflection assisted the surgeon in implanting the active-bending electrode array in the cochlea and minimizing buckling of the electrode array. The system also allowed a surgeon to monitor forces applied on the electrode array during insertion to insure that the inner ear was not injured, and the electrode array did not buckle.

### SUMMARY

In one aspect, the disclosed subject matter provides systems and methods for inserting a steerable array into an anatomical structure of the body. The system includes an insertion module for holding a proximal end of the steerable array. The system further includes a force sensor, at the proximal end of the steerable array, configured to detect force on the steerable array and to produce force information. The system further includes a position sensor configured to detect a position of the insertion module and to produce position information, the position information including a lateral position along an insertion axis and a first approach angle relative to a first reference axis. The system further includes a processor configured to receive the force information from the force sensor and the position information from the position sensor. The processor outputs performance information to a user. The performance information includes an indication of a first differential approach angle relative to an insertion path plan.

Embodiments of the disclosed subject matter may include one or more of the following features.

The insertion module may be a handheld device that is moved by the user. The handheld device may provide force feedback to the user based at least in part on an amplification of the detected force on the steerable array.

The insertion module may be adapted to be held and moved by a robotic device. The robotic device may be controlled by the user. The user may control insertion of the bendable array into the anatomical structure along the insertion axis, while the robotic device controls movement of the insertion module in directions other than along the insertion axis based at least in part on the insertion path plan. The robotic device may provide force feedback to the user based at least in part on an amplification of the detected force on the steerable array. The force feedback may be provided to the user through a telemanipulation unit that is manipulated by the user to control the robotic device.

The position information may further include a second approach angle relative to a second reference axis, the second reference axis being orthogonal to the first reference axis. The position information may further include a second lateral position along a second axis that is orthogonal to the insertion axis.

The force sensor may be further configured to detect moment on the steerable array and to produce moment information. An orientation sensor may be configured to detect an orientation of the insertion module to produce orientation information. The orientation information may include a roll angle of the insertion module relative to the insertion axis. The position sensor and the orientation sensor may be implemented as a pose sensor that detects position and orientation on the steerable array.

The performance information may include an indication of a differential insertion speed relative to the insertion path plan. The performance information may include an indication of a differential force on the steerable array relative to the insertion path plan. The performance information may include an indication of a differential insertion depth of the steerable array relative to the insertion path plan. The performance information may include an indication of safe insertion boundaries of at least one of insertion depth, insertion speed, approach angle, and force on the steerable array.

The processor may output a signal to stop insertion of the steerable array if at least one of insertion depth, insertion speed, approach angle, and force on the steerable array are outside of safe insertion boundaries. The safe insertion boundaries may include at least one of insertion depth, insertion speed, approach angle, and force on the steerable array are based at least in part on a statistical model of the anatomical structure.

The insertion path plan may be based at least in part on a model of the anatomical structure. The insertion path plan may substantially minimize expected force between the steerable array and the anatomical structure. The insertion path plan may be based on a model of the anatomical structure of a patient receiving the steerable array and substantially minimizes expected force between the steerable array and the anatomical structure of the patient. The insertion path plan may be determined to minimize force arising from contact between the steerable array and the anatomical structure.

The system may further include a bending actuator configured to bend an active-bending portion of the steerable array. The bending actuator may control the bending of the active-bending portion of the steerable array based at least in part on the insertion path plan. The bending actuator may control bending of the active-bending portion of the steerable array by displacing a thread connected to the active-bending portion. The thread may be connected to the active-bending portion so as to have an offset from a center axis of the steerable array.

The system may include a display unit for displaying the performance information to the user. The display unit may indicate a corrective action to the user based at least on the performance information. The indicated corrective action may include at least one of an insertion depth correction, an approach angle correction, an insertion speed correction, and a bending actuator displacement correction. The insertion module may induce vibration in the steerable array to reduce frictional force between the steerable array and the anatomical structure.

The insertion path plan may be determined by a method comprising minimizing a shape difference function, for each of a plurality of insertion depth values, to obtain a value of a bending actuator displacement and a value of the approach angle for each depth value. The shape difference function may be based at least in part on a shape model of the anatomical structure and a shape model of the steerable array. The shape model of the steerable array may be experimentally determined.

### DESCRIPTION OF DRAWINGS

The disclosed subject matter will be apparent upon consideration of the following detailed description, taken in conjunction with accompanying drawings, in which:

FIG. 1 is an anatomical depiction of a human ear and cochlear implant in accordance with some embodiments of the disclosed subject matter;

FIGS. 2A-B are detailed illustrations of cochlear implant system components and cochlear anatomy.

FIG. 3 demonstrates an active-bending electrode array during various ranges of deflection in accordance with some embodiments of the disclosed subject matter;

FIGS. 4A-B are depiction of systems for inserting an electrode array in accordance with some embodiments of the disclosed subject matter;

FIG. 5 is a diagram of a process for controlling systems for inserting an electrode array in accordance with some embodiments of the disclosed subject matter;

FIGS. 6A-B illustrate a four degree of freedom system and a scala tympani model, respectively;

FIGS. 7A-D illustrate various views of electrode array edge detection according to one embodiment;

FIGS. 8A-B illustrate calibration images of a steerable electrode array according to one embodiment;

FIG. 9 illustrates a kinematic model of a four degree of freedom robotic system according to one embodiment;

FIGS. 10A-B illustrate path planning configurations for various embodiments;

FIGS. 11A-B illustrate insertion simulations for two degree of freedom and four degree of freedom insertions, according to various embodiments;

FIG. 12 illustrates an experimental setup according to one embodiment;

FIG. 13 illustrates multiple steps in an experiment for steerable electrode insertion, according to one embodiment;

FIGS. 14A-B illustrate experimental results for two degree of freedom and four degree of freedom insertions, according to various embodiments;

FIGS. 15A-E illustrate problems with different degree of freedom arrangements;

FIGS. 16A-C illustrate multiple views of a steerable electrode array, according to one embodiment;

FIGS. 17A-C illustrate views of an experimental setup according to one embodiment;

FIG. 18 illustrates a vector diagram of angle and offset determination for an under-actuated robot, according to one embodiment;

FIG. 19 illustrates a vector diagram of inverse kinematics of an under-actuated robot, according to one embodiment;

FIG. 20 illustrates simulation results of bent electrodes, according to one embodiment;

FIGS. 21A-B illustrate graphical representations of path planning results, according to various embodiments;

FIG. 22 illustrates a graphical representation of spline results for an end of effector path for an under-actuated robot, according to one embodiment;

FIGS. 23A-B depict simulated insertion images and images taken during calibration of the electrode, according to one embodiment;

FIG. 24 illustrates various stages in simulations for two degree of freedom and four degree of freedom insertions, according to some embodiments;

FIG. 25 illustrates a graphical representation of simulated average angle and distance variations, according to some embodiments;

FIG. 26 illustrates an experimental setup, according to one embodiment;

FIG. 27 illustrates a graphical representation of certain insertion results under various experimental conditions;

FIGS. 28A-D illustrate insertion data from using a two degree of freedom system and a planar scala tympani model, according to some embodiments;

FIG. 29 illustrates insertion data from using a two degree of freedom system with ISFF and a three dimensional scala tympani model, according to some embodiments;

FIGS. 30A-B illustrate images taken during test insertions using a two degree of freedom system with ISFF and a three dimensional scala tympani, according to some embodiments;

FIGS. 31A-B illustrate insertion data using a four degree of freedom system without ISFF.

FIGS. 32A-B illustrate a digitization of the average distance and a quality of insertion metric, respectively, according to some embodiments;

FIG. 33 illustrates spline coefficients for one or more embodiments;

FIG. 34 provides a diagram showing static modeling of an electrode array, according to one embodiment;

FIG. 35 provides a diagram showing an electrode array contact pressure distribution, according to one embodiment;

FIG. 36 provides a graphical representation of an insertion simulation with various sensed force parameters, according tom some embodiments;

FIG. 37 illustrates an experimental setup according to one embodiment;

FIGS. 38A-D provide segmentation of insertion images, according to certain representations;

FIG. 39 illustrates various stages in an insertion process, according to one embodiment;

FIGS. 40A-C illustrate insertion force results with simulated results, according to certain embodiments;

FIG. 41 illustrates a logarithmic plot of insertion forces at a selected insertion speed, according to certain embodiments;

FIGS. 42A-C illustrate plots of average sensed forces at certain contact angles, measured at various selected insertion speeds, according to certain embodiments;

FIGS. 43A-D illustrate various embodiments of steerable electrode arrays with actuation based on an embedded offset Kevlar thread, according to one or more embodiments;

FIG. 44 illustrates a schematic representation of a steerable electrode with actuation thread, according to one or more embodiments;

FIGS. 45A-B illustrate digitized images and corresponding plot for different stages of insertion, according to one or more embodiments;

FIGS. 46A-B illustrate plots of an average distance metric and sensed force according to one or more embodiments;

FIGS. 47A-B provide images of calibration of the steerable electrode array according to certain embodiments;

FIG. 48. illustrates insertion simulation plots at various insertion depths, according to certain embodiments; and

FIG. 49 illustrates a flow chart showing an implant insertion path determination method according to certain embodiments.

### DETAILED DESCRIPTION

In accordance with the disclosed subject matter, electrode arrays and systems for inserting same are disclosed. The particular medical application for the steerable electrode arrays—cochlear implant electrodes—is of particular interest.

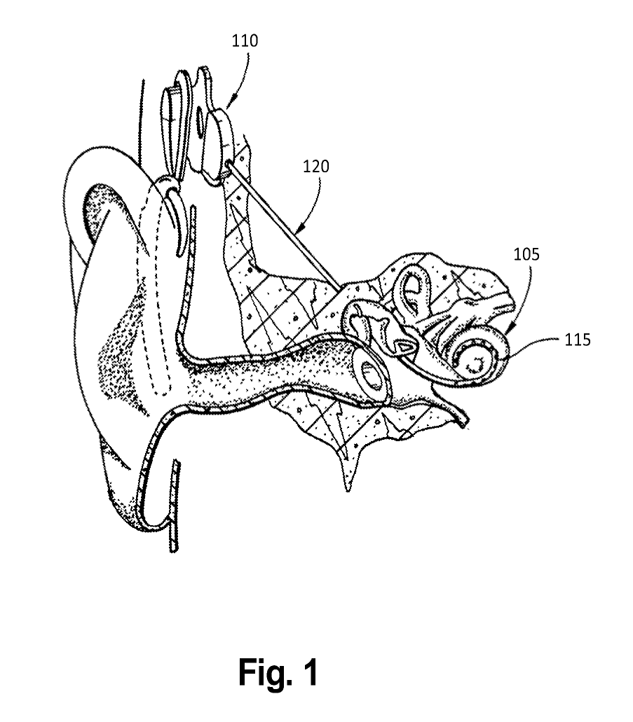

Cochlear implant surgery restores partial hearing for patients suffering from severe hearing loss due to damaged or dysfunctional neuroepithelial (hair) cells in the inner ear. The cochlear implant system includes a microphone, a signal processor, a transmitter, a receiver, and an electrode array, as shown in FIG. 1. This system converts the sound waves into electrical signals that are delivered to the auditory nerve through the implanted electrode array. The electrode array is implanted inside the scala tympani (though some earlier works explored insertion into the scala vestibuli).

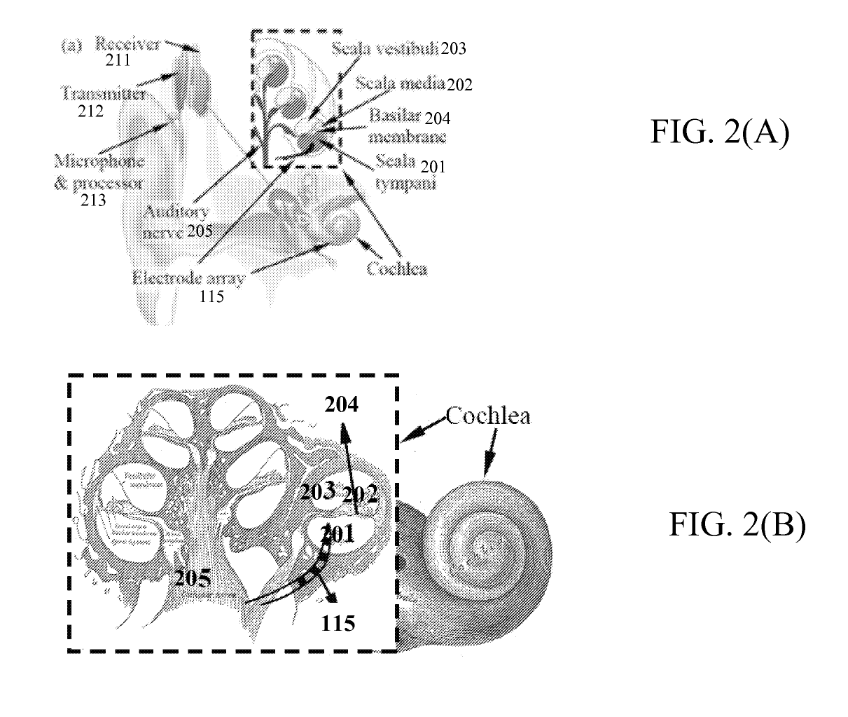

Referring to FIG. 1, an anatomical depiction of the human ear is displayed. It will be apparent that the disclosed subject matter can be used in other parts of the body (e.g., the lungs, heart, kidneys, fetus, etc.). For ease of understanding, this application primarily focuses on electrode arrays implanted in the inner ear 105. In some instances, a device 110 (e.g., transmitter, receiver, microphone, or processor) can be implanted under skin in a dimple carved into the mastoid bone and attached to an electrode array 115 located in inner ear 105 by a wire connection 120. For ease of reference, the inner ear shall refer to the cochlea, vestibule, and semi-circular canals described above with reference to FIG. 2B.

In certain studies by the inventors, a design of actively bent steerable electrode arrays was tested (see: Zhang, J., Xu, K., Simaan, N., and Manolidis, S., 2006, “A Pilot Study of Robot-Assisted Cochlear Implant Surgery Using Steerable Electrode Arrays,” Medical Image Computing and Computer-Assisted Intervention, 1 the entire contents of which are herein incorporated by reference). These steerable electrode arrays were actuated using an actuation wire embedded in a silicone rubber electrode array. More recently, inventors have worked under the supposition that the decrease in the insertion force of the electrode array will significantly reduce its buckling risk and the trauma rate during cochlear implant surgery. This supposition has been successfully used to define the insertion path planning and to experimentally compare different insertion strategies.

As noted above, using robot-assisted steerable array insertions can significantly reduce the insertion forces exerted upon the scala tympani, as compared to those generated by the insertion of non-steerable electrode arrays. The benefits of employing a steerable array—reduced insertion forces—can be improved yet further by refining the insertion process. The insertion process can be refined in several ways. First, the process can be improved by expanding the degrees of freedom in which the steerable electrode array can be moved. This enables improved positioning and maneuvering of the steerable electrode array during insertion in a complex insertion site. Second, the process can be refined by improved calculations for insertion path planning. Optimality measures that account for shape discrepancies between the steerable electrode array and the insertion site (e.g. the scala tympany) may be used to plan an insertion path that greatly reduces the forces generated at the insertion site and, subsequently the risk of damage to the surrounding tissue. These two advances—increases in degrees of freedom in which the electrode array may be effectively maneuvered and improved insertion path planning—are discussed in detail below.

By expanding the number of degrees of freedom available to refine motion during insertion, more successful insertion procedures can be achieved. In addition to steering the electrode array, it is possible to change its angle of approach with respect to the scala tympani. The approach angle may be changed in at least two directions relative to the insertion trajectory. The inventors have found by comparing steerable electrode array insertions using a two Degrees-of-Freedom (DoF) robot versus a four DoF robot, a four DoF insertion process is capable of superior outcomes to a two DoF insertion, when used for cochlear implants.

Insertion path planning strategies are presented in detail below for both two and four DoF insertions. Simulation results and experiments detailed below show that the four DoF insertions can improve over two DoF insertions. Moreover, changing the angle of approach can further reduce the insertion forces. The simulation results indicated below also provide the workspace requirements for designing a custom parallel robot for robot-assisted cochlear implant surgery.

In a pilot study on robot-assisted insertion of novel steerable electrode arrays (Zhang, J., et al.), steerable electrode arrays were actuated using an actuation wire embedded in a silicone rubber electrode array. This work showed that using steerable electrode arrays and robotic insertions can significantly reduce the insertion forces. Subsequently, nitinol shape memory alloy wires embedded inside the electrode array to provide steerability. Previous work used steerable electrode arrays and a two DoF robot that is capable of controlling the insertion depth and the bending (steering) of the electrode array. In addition to bending the electrode arrays, employed in this previous work, inventors have found it is possible to change its angle of approach with respect to the scala tympani, to achieve improved insertion results.

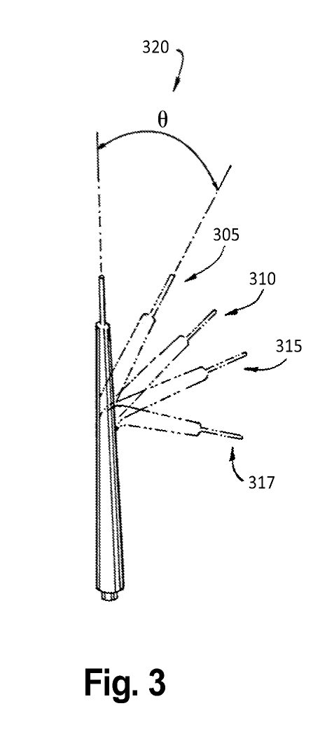

The steerable electrode array is depicted in FIG. 3. The steerable electrode array 300 is positioned in the inner ear 105 in relation to the anatomical structures described in detail with reference to FIGS. 1 and 2. Referring to FIG. 3, in some embodiments, n active-bending electrode array to create various amounts of deflection. For example, in certain embodiments, an actuation thread is used to control deflection. Applying tension to actuation thread (not shown) can create a substantial deflection in active-bending electrode array as is illustrated by deflections 305, 310, 315 and 317. As shown, in some embodiments, various angles of deflection 320 may be possible. For example, angles of deflection 320 may be in excess of 360 degrees in some embodiments. As described in greater detail below, the active bending and deflection of the electrode array can be refined and controlled with greater precision. Thus the active bending is not limited to the various angles of deflection 320 depicted above. Nor is the active bending limited to motion within a single plane, as illustrated simply in FIG. 3. The refined movement of the electrode array during insertion is the focus of the discussion below and the means by which inventors have substantially reduced risk of damage to cochlear anatomy during insertion of a cochlear implant system.

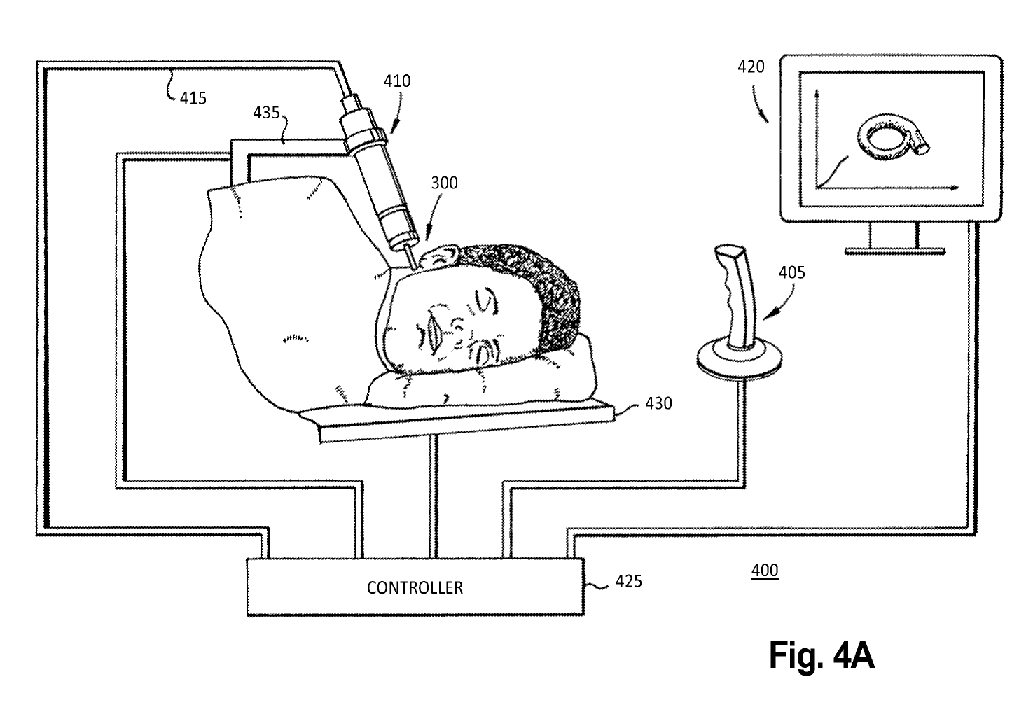

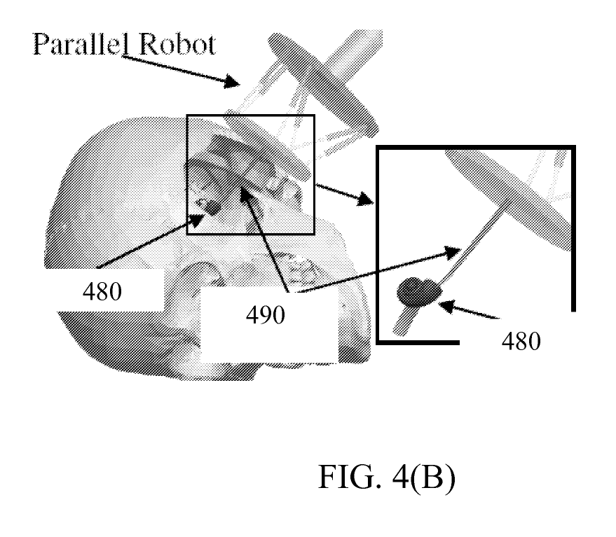

Referring to FIG. 4A, a system 400 can be used for inserting an electrode array (e.g., an active-bending electrode array or a passive-bending electrode array). System 400 can comprise an input device 405, an insertion module 410, a data connection 415, a controller 425, and a monitor 420. System 400 can also include a table 430 that allows motion in one or more directions (e.g., motion in a positive or negative direction along one or more orthogonal axes). In some embodiments, an arm 435 can connect insertion module 410 with table 430. In some embodiments, arm 435 can be robotic.

In use, insertion module 410 can be placed near the site of entry into the body (e.g., the ear canal, incision point, etc.). In some instances, insertion module 410 can sit on table 430 that is also located near the site of entry into the body. In some embodiments, insertion module 410 may be attached to a patient's head using a stereotactic frame or any other suitable mechanism. Using input device 405, the user can steer insertion module 410 into and inside the body. Insertion module 410 can then advance an electrode array into the body. While advancing, insertion module 410 can receive force and location measurements on the electrode array from sensors in insertion module 410. Force and location measurements can be displayed to the user on monitor 420. If an active-bending electrode array is used, controller 425 can deflect the active-bending electrode array by applying force (e.g., tension on an actuation thread) to the active-bending electrode array. When the electrode array is in a desirable position, insertion module 410 can be removed from the body leaving the electrode array in the body. In some embodiments, the angle of approach and deflection of an electrode array can be controlled by a path-planning module in controller 425, while the depth of insertion can be controlled through input device 405 by the user.

In some embodiments, insertion module 410 can reduce frictional forces on an electrode array by vibrating the electrode array. For example, insertion module 410 can vibrate an electrode array to decrease frictional forces as the electrode array traverses the inner ear. In some instances, vibration in insertion module 410 is a periodic oscillation, a-periodic oscillation, or a combination of both periodic and a-periodic oscillations. In some instances, vibration can be sensed by at least one sensor in system 400 and a counteractive force created by an at least one actuator located in insertion module 410.

In some embodiments, insertion module 410 can move in many directions. For example, insertion module 410 can have six-axis motion. Six-axis motion in insertion module 410 can be provided by a six-axis miniature parallel system. Further, insertion module 410 can have at least one sensor (e.g., an ATI Nano 17 U-S-3 six-axis force sensor produced by ATI Industrial Automation located in Apex N.C. or other suitable apparatus) for measuring force (e.g., force applied to an electrode array).

In some embodiments, system 400 guides an under-actuated active-bending electrode array. That is, system 400 has fewer actuators than degrees-of-freedom that can be controlled.

In some embodiments, rather than delivering an active-bending electrode array, system 400 delivers a passive-bending electrode array into the body. A passive-bending electrode array deflects when an external force (e.g., impacting tissue in the body) is applied to it.

In some embodiments, system 400 can incorporate a magnetic guidance system. In these embodiments, an active-bending electrode array comprises an active-bending portion, a passive-bending portion, and a magnet or a magnetic material. In some instances, there may be no actuation thread in the active-bending electrode array. A magnetic guidance system can be located external to the body. In some instances, a magnetic guidance system can be attached to insertion module 410. A magnetic guidance system can incorporate electro magnets. When a deflection is desired, the system can apply magnetic force to an active-bending electrode array and produce a deflection similar to that seen when force is applied by an actuation thread. In some instances, a magnet can be attached (e.g., by a thread) to insertion module 410. When desired, insertion module 410 can apply force and remove the magnet from the active-bending electrode array.

In some embodiments, input device 405 can incorporate force feedback. When force is detected on an electrode array (e.g., a force detected by an active-bending electrode array connected to the parallel robot through a small ATI Nano17 U-S-3 six-axis force sensor, or other suitable apparatus) force can be applied by input device 405 (e.g., Sidewinder Force Feedback™ from Microsoft Co., Impulse Stick from Immersion Corporation, or other suitable apparatus) to the user. For example, as force applied to an active-bending electrode array increases, input device 405 can vibrate or provide resistance with increasing strength indicating the situation to the surgeon.

In some embodiments, the surgeon controls the motion of the insertion module in all directions using the input device and relies on information displayed on monitor 420. For example, the surgeon can deliver an electrode array into the body and determine the safety of insertion based on, for example, the insertion force measurements provided on monitor 420 based on force feedback. Other types of performance information also may be displayed. For example, the display may provide an indication of how far the position of the insertion module has deviated from an insertion path plan. The display may also provide an indication of corrective action that can be taken to decrease the path differential. For example, the display may include indicator arrows, or other type of indicator, to direct the user to move the insertion module in a particular direction.

In some embodiments, the surgeon controls the insertion module in the axial direction during insertion while a controller 425 steers all other directions. In some instances, the controller, for example, has a preset path-planning module (or “insertion path plan”). In some instances, the preset path-planning module is based on, for example, 3D extensions of a 2D template of a cochlea. In some instances, using a path-planning module, the forces on the electrode array are reduced during insertion. In some instances, the surgeon controls the speed of the insertion (e.g., via the input device) while the controller controls the orientation of insertion and the bending of the electrode (e.g., using the insertion module).

In some embodiments, system 400 can perform the insertion automatically while offering the surgeon the possibility to take control. For example, the system may deliver an electrode array by following a path-planning module based on patient data. In some embodiments, monitor 420 can display the location of the active-bending electrode array in the body (e.g., the inner ear) and can also display a graph of the force being applied to the active-bending electrode array 300.

FIG. 4B illustrates a closer view of the insertion setup 400, according to certain embodiments. Insertion module 410 can comprise a parallel robot operable in response to surgeon control in parallel with a path-planning module based on patient data. In FIG. 4B, the positional relationship among the insertion module 410, the cochlea 480, and the active bending electrode array 300 during insertion is shown. In certain embodiments, the insertion apparatus is handheld, enabling the surgeon direct control over the insertion of the electrode array and removing the linkage of the controller 425.

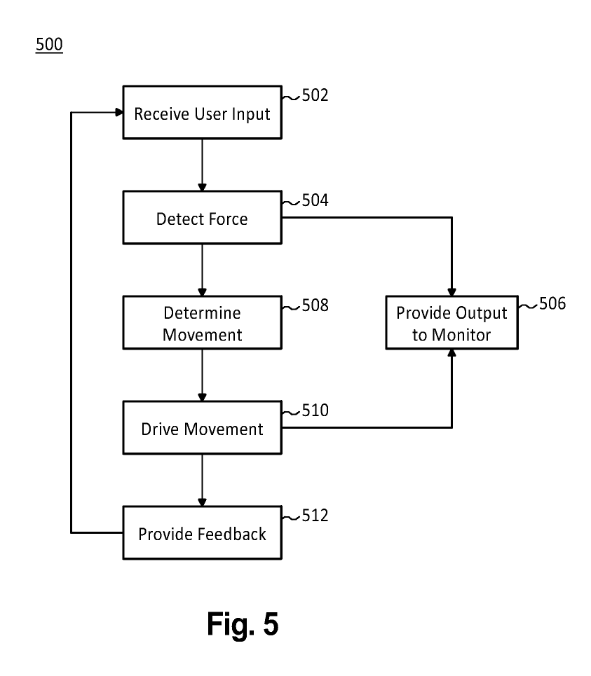

Turning to FIG. 5, a diagram of a process 500 that can operate in controller 425 is illustrated. As shown, process 500 can receive user input at 502. This user input may be provided from user input device 405 or insertion module 450, and may include hand movements (whether intentional or unintentional), button depressions, etc. At 504, process 500 can detect forces applied on an electrode array. These forces may be detected by insertion module 410 or 450 as described above. At 508, process 500 can determine the movement desired of insertion module 410 or 450. This movement can include movement to insert the electrode array, bend the electrode array, remove insertion module 410, remove hand tremors from insertion module 450, move arm 435, move table 430 or any other movement associated with insertion module 410, arm 435, table 430, and insertion module 450. The movement determined by 508 can include movement calculated by a path-planning module as described herein. At 510, process 510 may drive the movement of insertion module 410, arm 435, table 430, and insertion module 450. The drive signals may be generated by a suitable interface in controller 450. The force detected at 504 and the movement driven at 510 can be used to provide an output to monitor 420 at 506. At 506, process 500 can additionally or alternatively generate any other suitable output to monitor 420 as described herein, such as the various types performance information discussed below. At 512, process 500 may provide feedback to a user, such as by creating force on a joystick being used by the user, as described above. Process 500 may then loop back to 502. While the blocks of process 500 are illustrated in FIG. 5 as occurring in a specific order, it should be apparent to one of skill in the art that these blocks may occur in any suitable order or in parallel.

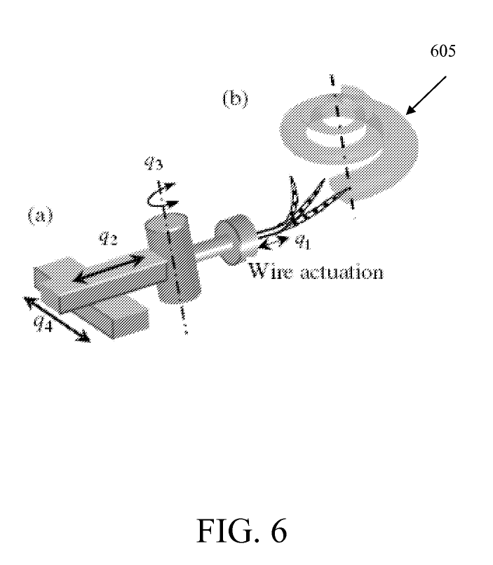

Referring to FIG. 6, an apparatus is schematically depicted for inserting the steerable electrode array into a cochlea 605 using four degree of freedom (4DoF) insertion methods. The four degrees of freedom are illustrated with reference vectors q₁, q₂, q₃, and q₄. Wire actuation for engaging the bending angle and curvature of the steerable electrode array is indicated by q₁. Advancing the steerable electrode array in, towards the cochlea, at the desired insertion depth, is controlled by motion in the direction indicated by q₂. Rotational motion for adjusting the approach angle of steerable electrode array as it enters the helical pathway of the cochlea is indicated by q₃. In addition, the motion orthogonal to the insertion direction of electrode array is indicated by q₄. Thus, in the case where four DoF are used, the angle of approach, the insertion depth, and the bending of the electrode array are all controlled during insertion. q₄ is depicted as linear movement which is result of the angular motion q₃. However, the depicted set up may also be used with a two DoF robot. In the case where only two DoF are used, only the insertion depth and the bending of the electrode array are controlled.

The inventors have found that changing the angle (q₃ and q₄) of approach of steerable electrode arrays greatly refines the insertion. Quantifying the importance of the changes in the angle of approach of the steerable electrode arrays is critical in achieving more successful insertion outcomes. Path planning algorithms are presented herein to provide the optimal bending (q₁) and the angle of approach (q₃ and q₄) of the steerable electrode array. These algorithms are simulated for determining the desired workspace of a custom-designed robot for steerable electrode array insertions. Further, experiments have been conducted to validate the efficacy of this approach. The mathematical modeling for the calibration and the insertion path planning of the steerable electrode array is provided. Below is a comparison between simulations of two DoF with four DoF electrode insertions. The experimental results for two and four DoF electrode insertions are also disclosed below, and the experimental results are found to confirm the simulations.

Two DoF and four DoF robot-assisted insertions of steerable electrode arrays for cochlear implant surgery can be refined by developing an insertion path plan. Active steering at multiple DoF can be implemented when the optimal placement path for the steerable electrode array is determined, relative to certain models of the implant site—e.g. the cochlea. In certain embodiments, an embedded strand in the electrode array provides an active steering Degree of Freedom (DoF). The calibration of the steerable electrode array and the insertion path plan for inserting it into planar and three-dimensional scala tympani models are discussed in detail below. The goal of the path planning is to minimize the intracochlear forces that the electrode array applies on the walls of the scala tympani during insertion. This problem is solved by designing insertion path planning algorithms that provide best fit between the shape of the electrode array and the curved scala tympani during insertion. Optimality measures that account for shape discrepancies between the steerable electrode array and the scala tympani are used to solve for the insertion path planning of the robot. Different arrangements of DoF and Insertion Speed Force Feedback (ISFF) have been simulated and experimentally validated, as shown below.

A quality of insertion metric describing the gap between the steerable electrode array and the scalar tympani model is presented, and its correspondence to the insertion forces is shown. The results of using one, two, and four DoF electrode array insertion setups are compared. The single DoF insertion setup uses non-steerable electrode arrays. The two DoF insertion setup uses single axis insertion with steerable electrode arrays. The four DoF insertion setup allows full control of the insertion depth and the approach angle of the electrode with respect to the cochlea while using steerable electrode arrays. It is shown below that using steerable electrode arrays significantly reduces the maximal insertion force (59.6% or more) and effectively prevents buckling of the electrode array. The four DoF insertion setup further reduces the maximal electrode insertion forces. The results of using ISFF for steerable electrodes show slight decrease in the insertion forces in contrast to a slight increase for non-steerable electrodes. These results show that further research is desired in order to determine the optimal ISFF control law and its effectiveness in reducing electrode insertion forces.

In certain embodiments, position sensing and orientation sensing for the steerable electrode array can be performed separately, in order to generate information from which the insertion metric(s) is calculated. In other embodiments, the position and orientation sensing functionality can be combined in a pose sensor which generates position and orientation information to be used in the calculations. In each case, the position and orientation sensors can report the position and orientation of the steerable electrode array relative to an inner surface of the scalar tymphani, relative to a reference point on the cochlea, relative to another anatomical reference point, relative to a reference point outside the patient, or any combination thereof.

In those embodiments in which the insertion system includes a hand-held insertion module, the position and orientation sensing may be performed by measuring inertial forces, employing a gyroscopic mechanism, or other mechanisms for spatial position and orientation sensing. In such a case, the position sensor performs at least one of a gyroscopic determination and an inertial determination to produce position information. Tracking mechanisms can be employed to gauge changes in position and orientation over time. Any of this information can be suitably used to evaluate the spatial position and orientation of the steerable electrode array and monitor the progress of the insertion.

In those embodiments in which the insertion system includes a robotic insertion module, the position and orientation sensing may be performed by the mechanisms outlined above. Alternately, the position and orientation sensing can be determined by tracking the movements of the robotic insertion module and calculating the position of the steerable electrode array relative to the starting (or a prior) position of the steerable electrode array. That is, if the insertion module is attached to a robotic device then the position information may be determined from the position of the various actuators of the robotic device based on control information received from the robotic device. Mechanisms for achieving such tracking of robotic devices are can be envisioned by one of skill in the art. In yet further embodiments, the aforementioned position and orientation sensing mechanisms can be used in tandem, generating position and orientation information that draws on more than one sensing source.

In certain embodiments, a stop signal mechanism can be employed. A stop signal mechanism may be implemented in order to avoid damaging contact between the steerable electrode array and the scala tympani, or other delicate parts of the anatomical structure. In certain instances, a stop signal can be used to override other insertion instructions to stop the insertion process before damage is effected. There are multiple ways to create and implement a stop signal mechanism and a few examples are now provided for the purposes of illustration. If the force sensed by a force sensor exceeds a certain permissible range (or a threshold is exceeded) the stop signal mechanism can be triggered. In some instances, this mechanism will relay information to the surgeon performing the insertion by generating a visual, audio and/or vibration cue that the surgeon will detect (i.e. warning message on the monitoring unit). In other instances, the stop signal mechanism will cause the processor to output amplified force information or an amplified feedback force. This amplified feedback force could be applied so that a surgeon using a handheld insertion module is able to detect the increase and stop the insertion. In other instances (e.g. robotic device—assisted insertion), the amplified feedback force could be expressed through a telemanipulation unit so that a surgeon performing the insertion will be able to detect the amplified force and stop the insertion. In yet other instances, the stop signal will cease the insertion process.

The stop signal mechanism need not be triggered by an actual force detected by a force sensor in the steerable electrode array. In some embodiments, the stop signal mechanism can be triggered when the position or orientation of the steerable electrode array deviates too far from the pre-selected path plan. For example, a stop signal mechanism can be triggered when position information exceeds a pre-selected position threshold or parameter, orientation information exceeds a pre-selected orientation threshold parameter, the speed of the insertion exceeds an upper or lower bound for permissible insertion speeds, etc. Yet other criteria to trigger a stop signal can be envisioned by one of skill in the art. In certain embodiments, the stop signal can trigger the insertion module to stop the motion at a lateral position along the insertion axis and at an approach angle relative to the first reference axis, at the time the processor outputs the stop signal.

The desirable thresholds, parameters, or upper and lower bounds for the aforementioned stop signal mechanisms can be determined in a number of ways. For example, the stop signals can be statistically determined from data gathered from numerous patients and expected likelihoods of damage to the scala tymphani can be computed for certain deviations from the planned path. In other instances, a generalized model of the scala tympani can be used as the source of the thresholds and parameters. In still further embodiments, the specific kinematic and static models disclosed below can be used to predict when damage to the inner ear will likely occur. From the kinematic and static models below, parameters or thresholds can be computed to minimize the likelihood of such damage. Whether statistical models derived from actual patient data or theoretical models derived from research into the structure of the cochlea are used, the stop mechanism can be a valuable means for avoiding trauma during insertions.

### Calibration and Path Planning of Two and Four DoF Insertions

### Image-Based Electrode Calibration

An optimal insertion path planning strategy reduces the intracochlear trauma and facilitates insertions. It can be assumed that reducing the insertion forces is correlated with decreased risk of electrode array buckling and electrode tip migration outside of the scala tympani. It therefore is desirable to minimize the shape discrepancy between the steerable electrode array and the scala tympani in order to minimize the desired insertion forces. In the discussion below, this approach is applied to four DoF electrode insertions.

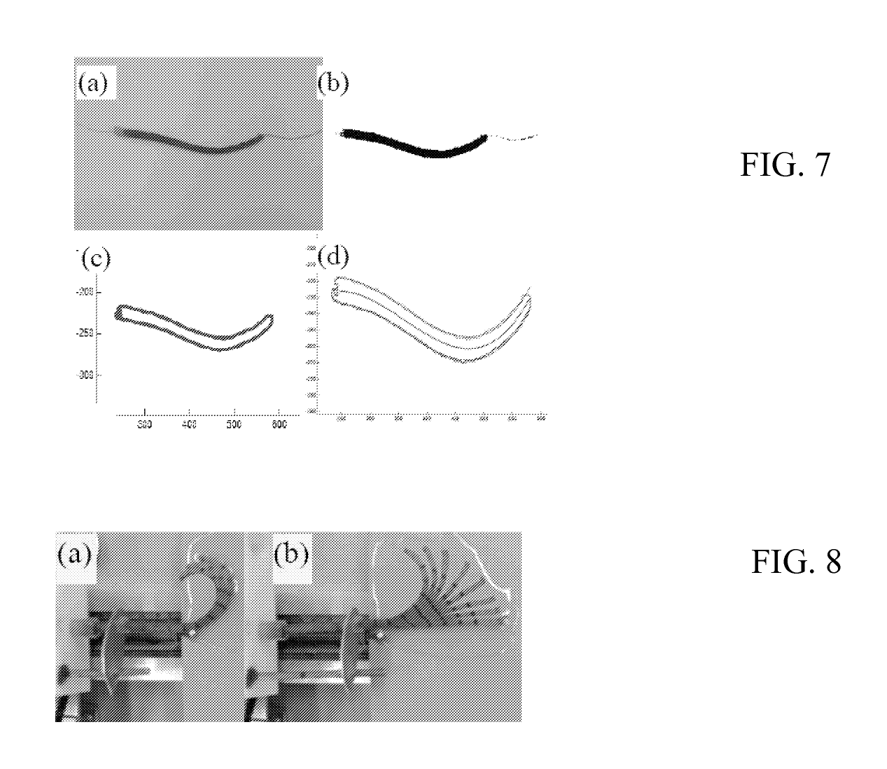

FIG. 7 illustrates a calibration process in which an automatic edge detection and curve fitting algorithm yields a closed-form approximation to the shape of the electrode array. FIG. 7A-D display different stages in an electrode array edge detection analysis, for any given bent shape of the steerable electrode array. FIG. 7A illustrates an original picture taken of the steerable electrode array. FIG. 7B illustrates the result when the original picture from FIG. 7A is converted to a black and white image. FIG. 7C illustrates the result when a Canny filter is applied to the black and white image from FIG. 7B to detect the edges of the electrode array. Finally, FIG. 7D illustrates the backbone curve fitting stage. In this embodiment, each edge is approximated by a fourth order polynomial curve and the backbone of the electrode array is calculated by averaging both edges.

FIG. 8 illustrates calibration images taken of the steerable electrode array, according to certain embodiments. FIG. 8A depicts the calibration images taken for a deep insertion of the steerable electrode array according to one embodiment. FIG. 8B depicts the calibration images taken for a shallow insertion of the steerable electrode array according to another embodiment. For any desired insertion depth, a series of pictures may be taken in order to calibrate a given steerable electrode array. This calibration process can be more generally articulated in the following manner. Picture j is associated with a given amount of pull q₁ⱼ on the actuation wire. For each picture, a polynomial model of the backbone of the steerable electrode array is fitted according to Eq. (1), below. In Equation (1), θ(s, q_{1,j}) represents the angle of the electrode array tangent at arc length s for q_{1,j}.

Vectors Ψ(s)=[1,s,s²,s³,s⁴]^{Γ}

aⱼ(q₁ⱼ)=[a_{j,0},a_{j,1},a_{j,2},a_{j,3},a_{j,4}]^{Γ}

represent the fourth-order polynomial approximation to the bending angle of the electrode array. To solve for the two parameter optimization problem, a least squares solution is applied in order to find the polynomial coefficients vector aⱼ(q_{1,j}). This result is a point: aⱼ(q_{1,j})ε [symbol] for each j(j=1, 2, . . . , z).

$${θ}_{j} ( s , {q}_{1 j} ) = {\sum }_{p = 0}^{4} {a}_{j , p} {s}^{p} = {ψ}^{T} ( s ) {a}_{j} ( {q}_{1 j} ) j = 1 , 2 , … , z \qquad ( 1 )$$

Next, a second interpolation over vectors aⱼ(q_{1,j})'s is carried out using cubic splines. Following known mathematical expressions, inventors define k intervals (segments) [aₖ(q_{1,k}), aₖ₊₁(q_{1,k+1})] where k=1,2, . . . , z−1. For any given q₁ε[q_{1,k}, q_{1,k+1}], Eq. (2) below, solves for the coefficients of the cubic spline for segment k; then the shape of the electrode is given by Eq. (3), where cₖ(q₁) and Bₖ are given by Eqs. (4) and (5). In Eq. (4), a′ₖ denotes the derivative of aₖ(q₁) with respect to q₁.

$$\begin{matrix}{a}_{k} ( {q}_{1} ) = {B}_{k}^{T} {c}_{k} ( {q}_{1} ) & ( 2 ) \\ {θ}_{k} ( s , {q}_{1} ) = {ψ}^{T} ( s ) {B}_{k}^{T} {c}_{k} ( {q}_{1} ) \\ {q}_{1 j = k} \leq {q}_{1} \leq {q}_{1 j = k + 1} , 1 \leq k \leq z - 1 & ( 3 ) \\ {c}_{k} ( {q}_{1} ) = {[ 1 , {q}_{1} , {q}_{1}^{2} , {q}_{1}^{3} ]}^{T} & ( 4 ) \\ {B}_{k} = [ \begin{matrix}1 & 0 & 0 & 0 \\ 0 & 1 & 0 & 0 \\ - 3 / {q}_{{1}_{k + 1}}^{2} & - 2 / {q}_{{1}_{k + 1}} & 3 / {q}_{{1}_{k + 1}}^{2} & - 1 / {q}_{{1}_{k + 1}} \\ 2 / {q}_{{1}_{k + 1}}^{3} & 1 / {q}_{{1}_{k + 1}}^{2} & - 2 / {q}_{{1}_{k + 1}}^{3} & 1 / {q}_{{1}_{k + 1}}^{2}\end{matrix} ] [ \begin{matrix}{a}_{k} \\ {a}_{k}^{′} \\ {a}_{k + 1} \\ {a}_{k + 1}^{′}\end{matrix} ] & ( 5 )\end{matrix}$$

### Insertion Path Planning

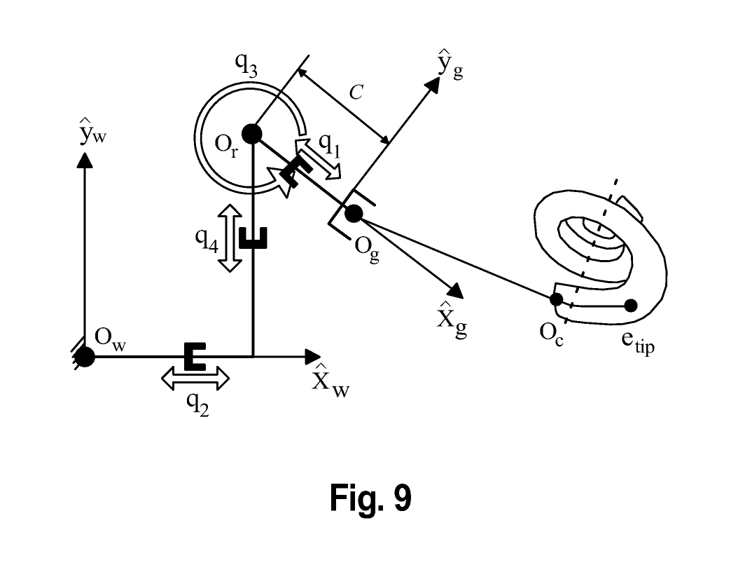

The following description focuses on the path planning for four DoF electrode insertions. In FIG. 6, the approach angle of the electrode array with respect to the scala tympani is denoted by q₃ and the electrode actuation wire pull is denoted by q₁. The translation components of the gripper holding the electrode array are given by q₂ and q₄. A kinematic model of the robot and the electrode array is illustrated in FIG. 9. For any given insertion depth d, the optimal values of q₁ and q₃ are obtained by minimizing the objective function in Eq. (6). This function minimizes the shape discrepancy between the steerable electrode array and the scala tympani model using interpolation of the experimental data results to find the global minimum. L represents the overall length of the electrode array, and θ_{c}(s_{c}) stands for the angle of the curve tangent of the scala tympani at arc length s_{c}.

$${arg min}_{{q}_{1} , {q}_{3}} {\int }_{L - d}^{L} {( θ ( τ , {q}_{1} ) - {θ}_{c} ( τ - ( L - d ) ) + {q}_{3} ( d ) )}^{2} d τ \qquad ( 6 )$$

Once q₁ and q₃ are found, the desired translation DoF q2 and q4 are easily found by Eq. (7) using the inverse kinematics of the robot in FIG. 9.

$$[ \begin{matrix}{q}_{2} \\ {q}_{4}\end{matrix} ] = {o}_{g} ( {q}_{1} , {q}_{3} ) - [ \begin{matrix}c cos {q}_{3} \\ c sin {q}_{3}\end{matrix} ] \qquad ( 7 )$$

where c is the length from o_{g} (position of the robot gripper) to or (rotation center).

### Simulation and Workspace Determination

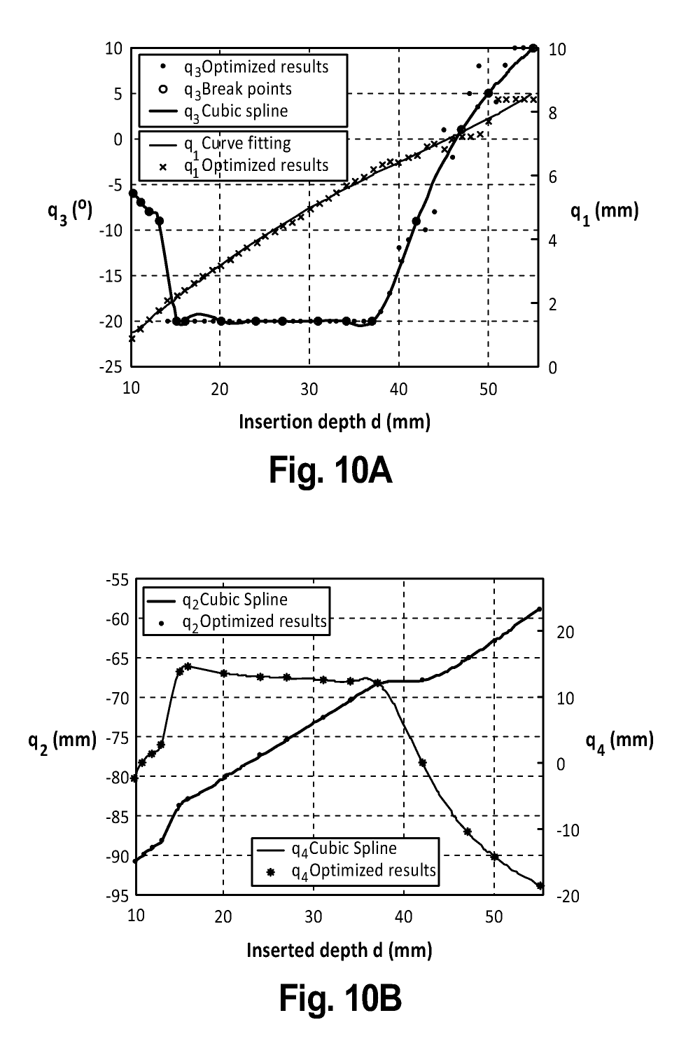

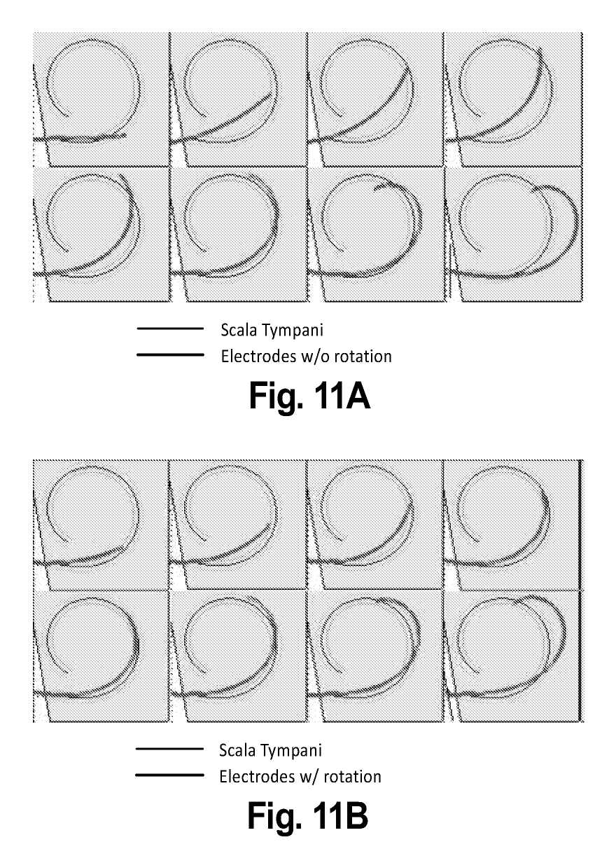

FIGS. 10A-B. show the simulation results of insertion path planning for four DoF insertions using the steerable electrode array of FIG. 8. In FIG. 10A, the path planning for the electrode actuation wire pull q₁ (bending) and approach angle with respect to the scala tympani q₃ are shown as functions of insertion depth. In FIG. 10B, the translation components of the gripper holding the electrode array including advancement into the scala tympani q₂ and lateral motion controlling the approach angle q₄ are shown as functions of insertion depth. FIG. 11 compares the two DoF with four DoF insertions based on these simulation results. In FIG. 11A, a simulation for a two DoF insertion is shown (electrodes without rotational motion) in relation to the scala tympani. In FIG. 11B. a simulation for a four DoF insertion is shown (electrode with rotational motion) in relation to the scala tympani. Visual comparison between FIGS. 11A and B reveals that the four DoF insertions fit the shape of the scala tympani better than the two DoF insertions. This result is analytically confirmed by experiments detailed below. From FIGS. 10A-B, the desired robot joint values vary within the intervals:

q₃ε[−20°,10°],q₂ε[−91,−58]mm, and q₄ε[−18,15]mm.

The desired joint ranges are thus 33 mm, 30°, and 33 mm for q₂, q₃, q₄ respectively. If scaled down properly, these results provide the desired workspace for robot-assisted insertion of true:size (1:1) steerable cochlear implant electrode arrays.

In the experimental setup depicted in FIG. 8, the distance between the revolute joint of the robot oᵣ and the electrode tip eₜᵢₚ for the 3:1 electrode array is 110 mm. In real applications for cochlear implants, the estimated depth of full insertions is about 27 mm measured from the cochleostomy point o_{c} to the electrode tip eₜᵢₚ. Yet other measurements are envisioned to suit the particular anatomy and applications desired, as will be evident to one of skill in the art. It is expected that the average distance from the cochleostomy point o_{c} to the opening in the skull is 60 mm for adults. For the purposes of the present embodiments, inventors increase this number by 10 mm for safety. Hence the desired distance from or to eₜᵢₚ of a true size (1:1) steerable electrode array is estimated to be 97 mm.

Since the robot rotates the electrode while keeping the cochleostomy point o_{c} fixed during insertion, it is easy to scale down the workspace of the robot. In certain embodiments, the scaling can be done while considering the electrode array as a rigid body rotating about o_{c}. Since the distance between oᵣ and o_{c} varies during insertion, inventors choose to use the full length of the electrode to provide a conservative estimate for the desired workspace (i.e. inventors use the condition o_{c}=eₜᵢₚ). To scale down the workspace for a 1:1 steerable electrode, a scaling factor of 97/110 is directly applied to the translational joints of the robot. The resulting desired joint ranges are 30 mm for q₂ and q₄. The minimal expected rotation range for joint q₃ is not scaled down and it remains 30°.

Inventors have found it beneficial to use this workspace estimation to design a miniature Stewart-Gough parallel robot for electrode array insertion. In certain embodiments, it is desirable to implement design goals for this robot of ±20° tilting about any axis and ±20 mm translation in all directions. For example, in the experimental validation discussed below the parallel robot type was chosen for its compactness, precision, and portability.

### Experimental Validation

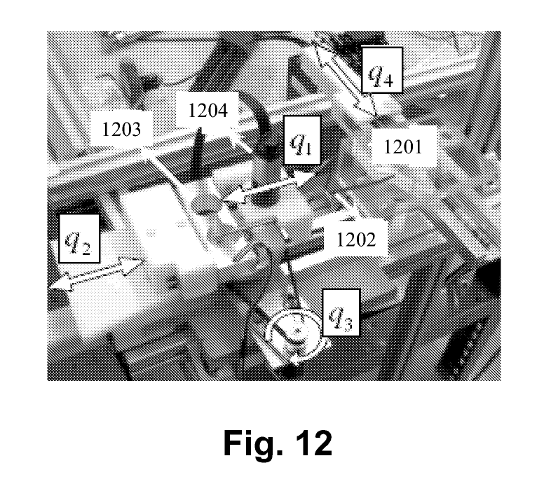

As noted above, FIG. 11 shows that changing the angle of approach of the electrode array with respect to the scala tympani allows decreasing the shape discrepancies between the steerable electrode array and the scala tympani. These simulation results were based on the calibration and the simulation path planning for the steerable electrode in FIG. 8. However, one of skill in the art will appreciate that certain modifications to the calibration and simulation path planning may be made for certain embodiments of the steerable electrode. In the present example, the same steerable electrode was used for two DoF and four DoF insertion experiments using the experimental setup of FIG. 12. Specifically, the experimental setup of FIG. 12 includes the planar scala tympani model 1201, the steerable electrode array 1202, the single axis force sensor 1203 and the motor for the steerable electrode array 1204. In this particular experiment, inventors have validated the correlation between the results in FIG. 11 and the reduction of the electrode insertion forces when four DoF insertion path planning is used compared to only two DoF insertions.

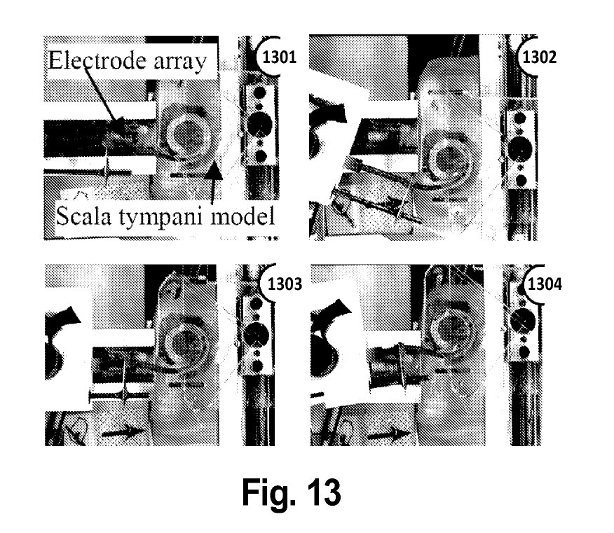

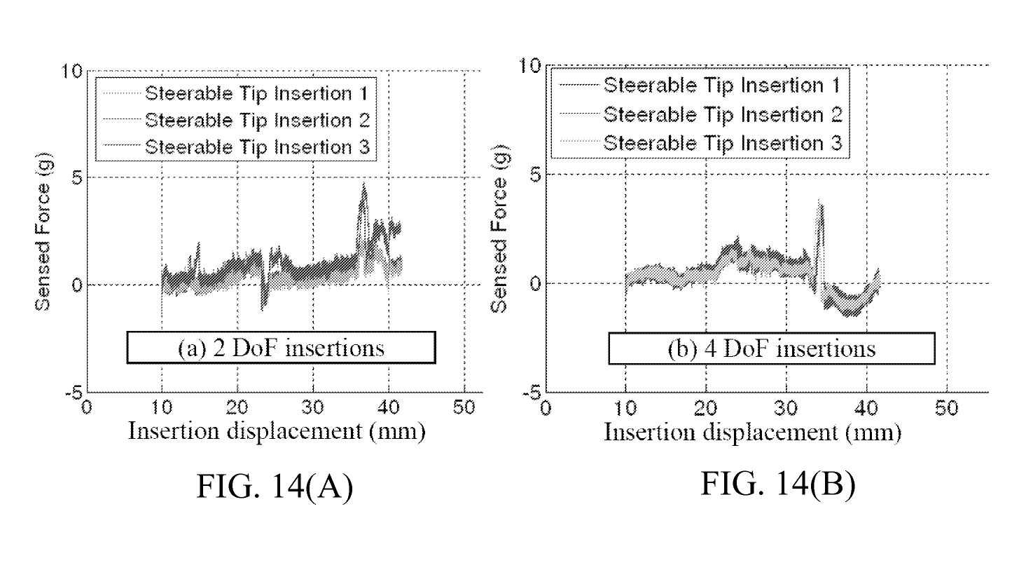

During these particular experiments, the scala tympani channel was wetted by glycerin to emulate the environment inside the cochlea. The robot of FIG. 12 was controlled with Linux Real Time Application Interface (RTAI) with a closed loop rate of 1 kHz. An AG NTEP 5000d single axis force sensor was used in the setup to measure the axial insertion force of the steerable electrode array during the whole insertion process and it was capable of detecting ±0.1 g force using a 13 bits A/D acquisition card. One of skill in the art will understand this example to be illustrative and not exclusive. Each insertion was repeated, in this case, three times to validate the repeatability of the results. FIG. 13 illustrates how the approach angle of the electrode array can be changed with respect to a planar scala tympani model during four DoF insertions. Specifically, FIG. 13 illustrates the electrode array with respect to the scala tympani model at a first phase 1301 of insertion, a second phase 1302 of insertion, a third phase of insertion 1303 and a second phase of insertion 1304. Each image demonstrates a different positioning of the electrode array, as a result of the multiple directions of motion available. The force reading data of these experiments are shown in FIG. 14.

FIG. 14A shows the results of two DoF steerable electrode array insertions. In the present embodiment, the average insertion force for the three experiments is 0.7 grams. For the purposes of comparison, the four DoF insertions in FIG. 14B had an average insertion force of 0.40 grams, 43% smaller than two DoF robot insertions. The peak of force happens when the electrode array first hits the outer wall of the scala tympani model and therefore generates the maximal insertion force. According to the present embodiment, for two DoF insertions, the maximal peak value among three experiments is 4.8 grams. In this embodiment, four DoF insertions keep the peak value as 3.9 grams, 19% less than the two DoF insertions peak. Accordingly, inventors have found that the negative forces happen in FIGS. 14A-B because the steerable electrode hugs the inner wall of the scala tympani. These results suggest the effectiveness of adjusting the angle of approach of the steerable electrode array with respect to the scala tympani in reducing the insertion forces.

### Insertion Path Planning

As noted above, the kinematics, calibration, and insertion path planning are important for safe insertion of steerable electrode array into a given 3D cavity (scala tympani inside the cochlea). Inventors have identified the desirability of a mechanism of safe electrode array insertions in cochlear implant surgery and have identified a number of insertion errors. Thus the following section specifically identifies insertion errors and discloses optimal insertion path planning methods for correction of these insertion errors in cochlear implant surgery.

Most broadly, the problem of inserting flexible under-actuated objects into human anatomies is important for safe catheter insertion, neurosurgery, endovascular surgery, colonoscopy, etc. These related problems associated with inserting flexible, under-actuated objects into human anatomies highlight the importance of correcting insertion errors in specific applications.

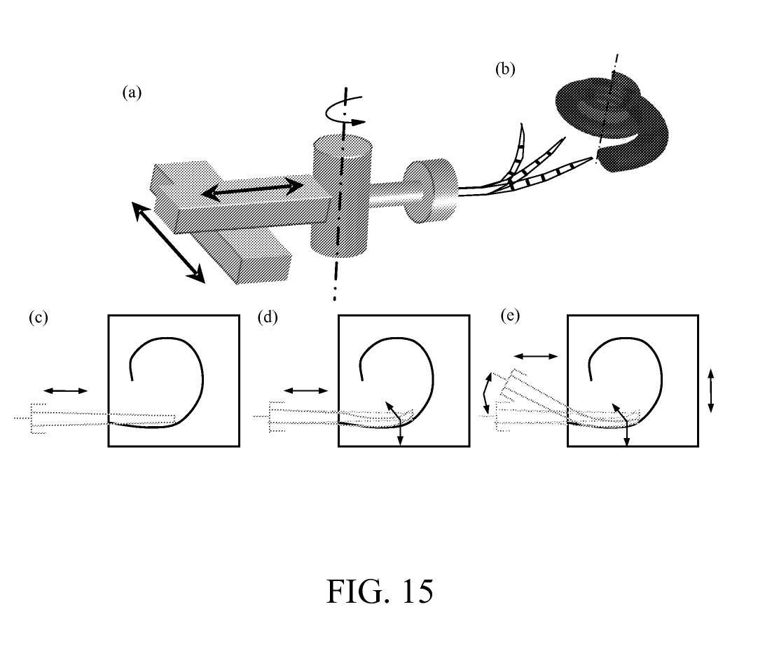

Referring to FIG. 15, inventors have identified certain problems with different arrangements of cochlear implant insertion systems. FIGS. 15A-B depict the insertion apparatus discussed above with reference to FIG. 6. in the case in which an under-actuated steerable electrode array assumes a predetermined 3D minimal energy shape when actuated. FIG. 15C illustrates the problem of a single DoF insertion, in which the electrode may be moved into the helical cavity of the cochlea but is not controllable in any other degree of freedom. FIG. 15D illustrates the problem of a two DoF insertion in which the electrode may be moved into the helical cavity of the cochlea and actuated to enable curvature of the electrode (steerable) itself but is not controllable in other degrees of freedom. FIG. 15E illustrates the case of a four DoF insertion in which the approach angle may be controlled in two directions, in addition to the aforementioned DoFs. Problems may potentially arise with this four DoF when the path of motion in each degree of freedom is insufficiently planned. The calibration of the steerable electrode array and the insertion path planning for safe insertion are presented below.

Inventors have found that an optimal insertion significantly reduces the insertion force of the electrode array. This optimality criterion is used because increased insertion forces are directly related to the increased risk of buckling of the electrode array inside the scala tympani. Buckling of the electrode array during insertion is very likely to result in trauma to surrounding anatomies. Therefore, the optimality criterion are derived to reduce the risk of the electrode buckling inside the scala tympani and consequently reduce the risk of trauma to the cochlea.

Due to the small size of the steerable electrode array, one can assume that controlling its shape is limited to using a single actuator. The importance of three additional degrees of freedom (DoF) desired for the steerable electrode array is evaluated through simulations and experiments. Inventors compare the insertions using one DoF system (FIG. 15C), two DoF system (FIG. 15D) and four DoF system (FIG. 15E). Discussed in detail below are simulated average angle and distance variations as an optimality measure which are then correlated to the shape similarity between the steerable electrode array and the scala tympani.

This differs notably from previous works on flexible object insertions focused mainly on inserting flexible beams into straight holes, modeling and path planning for flexible object manipulation, and robot-assisted insertion of steerable catheters. For example, in certain work, the deflection of the beam has been depicted by applying former large deflection theory in uniformly distributed load. Two cases have been addressed: insertion with loose tolerance, and insertion with a tight tolerance. It was found that the robot gripper only needs to follow the shape of deflected beam during the insertion for the loose clearance case. The tight-clearance case desired modifying the position of the robot gripper along the involute of the beam curve prior to reattempting insertion by following the deflected beam curve. Inventors have found that the problem of safe insertion of a flexible object into a 3D cavity has not been appropriately addressed.

The present disclosure addresses the problem of safe insertion of a flexible under-actuated object into a curved cavity instead of a simple hole. A general path planning algorithm for inserting under-actuated steerable electrode array into a curved cavity (scala tympani) is provided to achieve the desired safe insertion. The disclosed approach is explained below along with its relevance to cochlear implant surgery. The importance of changing the end conditions of the flexible object as opposed to only controlling its steerable portion are compared by simulation and verified by experiments. A physically meaningful optimality measure is defined and correlated to the desired insertion forces obtained from the experimental results.

In the discussion below, the kinematic modeling of a robotic system inserting a steerable electrode array and its calibration are provided. A modal approach to determine the shape of the steerable electrode array and solve for the insertion path planning of the steerable electrode array is applied. The calibration and simulations of the insertion process and comparison between systems with different DoF are presented, as are the experimental setup and experimental results using one, two and four DoF systems. A comparison of the insertions into planar and 3D cavity models (scala tympani) with and without ISFF is also provided. The explanation for the effectiveness of the path planning algorithm of the flexible robot is detailed below.

### Kinematic Modeling

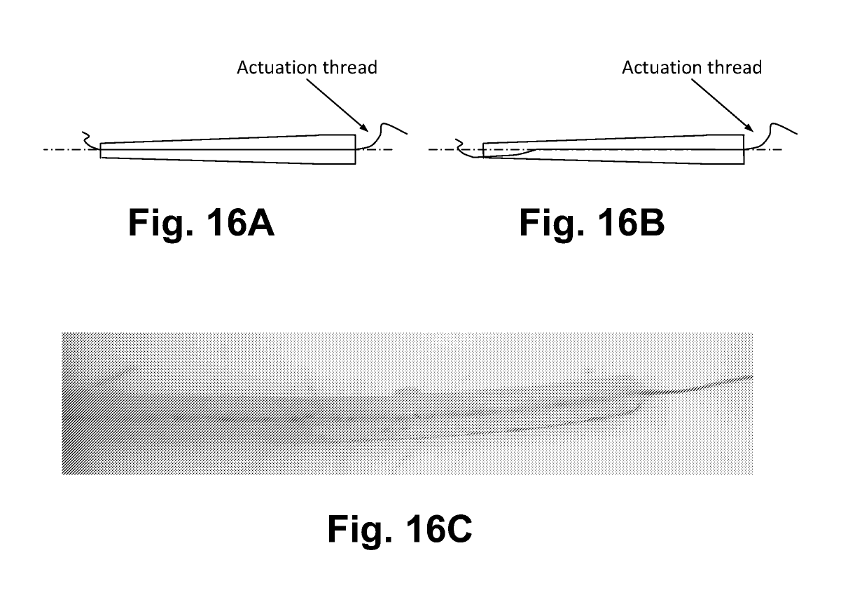

FIG. 16 shows the conceptual design of the steerable electrode array for cochlear implant surgery. Different from the commercial electrode arrays, the steerable electrode array of the present embodiment has a Kevlar strand embedded inside. This is shown in the schematic representations of FIGS. 16A and B. FIG. 16A provides a side view while FIG. 16B provides a top view. The strand is offset from the center of the electrode array and it is fixed at its tip, as seen in FIG. 16B. When the strand is pulled at the base, different bent shapes are obtained. Considering the fabrication cost to make a real electrode, inventors first fabricated a 3:1 scaled up steerable electrode array based on MedEl electrode. This physical model is shown in the image in FIG. 16C.

### Scala Tympani Model

The 2D template of the scala tympani model was first provided by Cohen (Cohen, L., Xu, J., Xu, S. A., Clark, G. M., 1996, “Improved and Simplified Methods for Specifying Positions of the Electrode Bands of a Cochlear Implant Array,” The American Journal of Otology, 17, the entire contents of which are herein incorporated by reference) to aid surgeons with an estimation of the insertion angle. Based on his model, a scaled up (3:1) planar scala tympani model is made. This model provides insertion angle up to 340 degrees, which is understood to be sufficient to demonstrate the effectiveness of using steerable electrode arrays because buckling in the un-actuated case can be avoided with active steering.

Models of the scala tympani have been discussed in previous studies which have provided three-dimensional modeling of the scala tympani. Internet-based 3D visualization tools for the cochlea based on a 3D generalization of Cohen's 2D spiral template have also been created. The backbone curve of the scala tympani is given by Eq. (8) below where r, Z and θ are the cylindrical coordinates of this curve (r is the radial distance to the curve, Z is the height, and θ is the angle). The values of the constants a, b, c, d, θ₀, p are based on known models of the cochlea. The 3D scala tympani model employed here has a fixed angle helix, which leads to a simple solution of the insertion angle. The cross section of the scala tympani may be modeled by an ellipse according to dimensions below.

$$θ ε [ 10.3 ° , 910 ° ] , \\ R = \{ \begin{matrix}c ( 1 - d log ( θ - {θ}_{0} ) ) & θ < 100 ° \\ a {e}^{- b θ} & θ \geq 100 °\end{matrix} \} , \\ z = p ( θ - {θ}_{0} ) \qquad ( 8 )$$

The shape of the planar bent electrode array is characterized by θₑ(s,q₁), where θₑ is the angle at arc length s along the backbone of the electrode array given the actuation of the strand q₁. s=0 represents the base of the electrode array and s=L denotes the tip of the electrode array. Let the minimal energy solution for the direct kinematics of the electrode array be approximated using a modal representation in Eq. (9), where a is the vector of modal factors.

θₑ(s,q₁)=Ψ(s)^{T}a(q₁),a,Ψε [symbol] (9)

where Ψ(s)=[1,s,s², . . . , sⁿ⁻¹]^{T}. Further denote the modal factors by

a(q₁)Aη(q₁) (10)

where ηε [symbol] , Aε [symbol] , η=[1, q₁, q₁², . . . , q₁^{m-1}]^{T}. For high-order polynomial approximations (m>6), a set of orthogonal polynomials (e.g. Chebyshev polynomials) should be used for considerations of numerical stability. Through experimental digitization, the shape of the electrode array is digitized by r equidistant points along its backbone in z different images of the electrode array associated with z different values of q₁ (the amount of pull on the actuation strand). The digitization results are stored in the experimental data matrix Φε [symbol] , where Φ_{i,j}=θₑ(sᵢ, q₁ⱼ) Using the modal representation in Eq. (9), the experimental data matrix is expressed by Eq. (11).

$$Φ = \underbrace{[ \begin{matrix}{ψ}^{T} ( s = 0 ) \\ ⋮ \\ {ψ}^{T} ( s = {s}_{max} )\end{matrix} ]}_{Ω} {A}_{n \times m} \underbrace{[ \begin{matrix}η ( {q}_{1} = 0 ) , … , \\ η ( {q}_{1} = {q}_{1 max} )\end{matrix} ]}_{Γ} = {Ω}_{r \times n} {A}_{n \times m} {Γ}_{m \times z} \\ where Ω = {[ \begin{matrix}1 s = 0 & ⋯ & s = {0}^{n - 1} \\ & ⋮ & \\ 1 s = {s}_{max} & ⋯ & s = {s}_{max}^{n - 1}\end{matrix} ]}_{r \times n} \\ and Γ = {[ \begin{matrix}1 & & 1 \\ {q}_{1} & … & {q}_{1 max} \\ ⋮ & & ⋮ \\ {q}_{1}^{m - 1} & & {q}_{1 max}^{m - 1}\end{matrix} ]}_{m \times z} \qquad ( 11 )$$

are Vandermonde matrices corresponding to the r numerical values of s and the z values of q₁ used to generate the experimental data matrix Φ. Solving Eq. (11) for the electrode array calibration matrix A, provides the desired solution for the direct kinematics problem. The solution of this algebraic matrix equation is given by [Γ^{T} [symbol] Ω] Vec(A)=(Φ). Where [symbol] represents Kronecker's matrix product and Vec(A m×n)=[a₁₁ . . . aₘ₁ . . . aₘ₂, . . . , a₁ₙ . . . aₘₙ]^{T}.

### Electrode Array Calibration

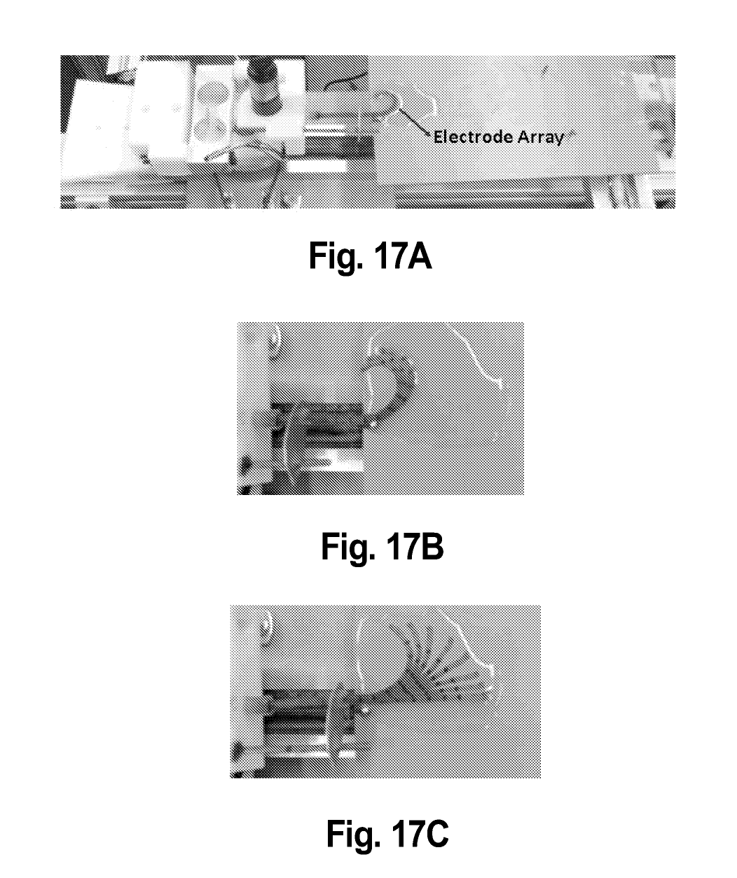

As described in the above section, the shape characteristics of the electrode array are fully expressed by the calibration matrix A, which is solved experimentally. The calibration setup of the electrode array is shown in FIG. 17A. FIG. 17B shows the calibration process for a shallow insertion depth with a supporting ring position. FIG. 17C shows the calibration process for a deep insertion depth with the supporting ring position. In the calibration process, only the joint for pulling the actuation strand of the electrode array is active. The electrode is placed on a platform with glycerin in between to reduce the friction between the electrode and the supporting platform.

Since the electrode array is long and subject to buckling, a support ring is needed to prevent this failure mode at the unsupported portion of the electrode array outside the scala tympani model. Although the position of the support ring can be continuously changed during the insertion, inventors chose to place the ring in an extended position during shallow insertions and in a retracted position during deep insertions, FIG. 17B and FIG. 17C. In each picture, the bending angles at each marking point are recorded. Then the experimental data matrix Φ is calculated from the calibration figures. yielding the calibration matrix A by the algebraic matrix equation, Eq. (11).

### Insertion Path Planning for Robotic Electrode Insertion

An algorithm is applied to solve for the shape, orientation and position of the inserted and bent part of the electrode array such that it can best approximate the curved shape of the scala tympani. This approximation meaningfully assumes that that when the shape of the bent electrode array matches the curve of scala tympani, the insertion will be proceed with less force than a geometrically unmatched array. In order to find the best shape at each insertion depth, the optimization problem is solved by finding the optimal paths for the steerable electrode array and a11 three additional DoF of the insertion unit.

The anatomical 3D scala tympani model based on Eq. (8) has a constant helix angle. Since the steerable electrode array used in the experimental setup is designed to bend in plane, it is tilted about its longitudinal axis by an angle equal to the helix angle of the scala tympani. This simplifies the insertion path planning and the electrode array design and fabrication. Hence, the inventors have found that the optimal insertion path planning is achieved based on the planar scala tympani model.

### Insertion Path Planning

The problem of insertion path planning includes finding the optimal orientation and position of the base of the electrode array and the optimal steering of its tip in order to minimize intra-cochlear damage during insertion. As noted above, FIG. 15A shows the kinematic layout of a four DoF robot comprised of an insertion unit and a steerable under-actuated electrode array. Specifically, FIG. 15A illustrates the under-actuated robot. FIG. 15B illustrates the known 3D helical cavity which models the cochlea. FIG. 15C shows the single DoF insertion. FIG. 15D shows the two DoF insertion and FIG. 15E shows the 4DoF insertion. The insertion unit is a 3 DoF planar robot that allows adjusting the angle and the offset of the electrode array with respect to the scala tympani.

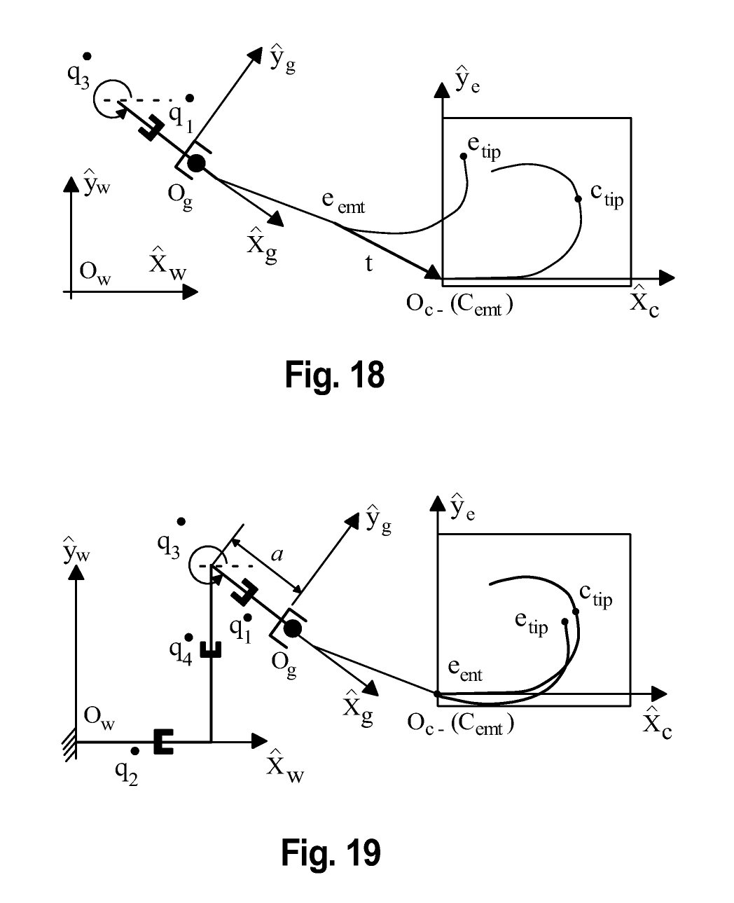

FIG. 18 shows an electrode array that is optimally bent and rotated in plane in order to fit the shape of the scala tympani for a given insertion depth. Frames {w}, {g}, {c} designate the world coordinate system, the robot gripper coordinate system, and the cochlea coordinate system. q₁* designates the optimal amount of retraction of the actuation strand of the steerable electrode array. q₃* denotes the optimal rotation of the robot gripper. cₜᵢₚ is the point on the centerline of scala tympani which corresponds to the tip of the inserted electrode array eₜᵢₚ. eₑₙₜ is the point on the center line of the electrode array which corresponds to the entrance of the scala tympani cₑₙₜ.

### Desired Orientation Determination

Once the electrode array calibration matrix A is generated, for any given q₁, \tilde{θ}₁(s) yields a column vector of Φ(s, q₁) that represents the shape of the bent electrode array. Similarly, the shape of scala tympani can be defined as \tilde{θ}_{c}(s_{c}), where s_{c}ε[0, L_{c}] is the arc length along the central curve of the scala tympani model. The insertion depth d is defined by the arc length of the inserted part of the electrode array. The objective function for desired angle determination is given by Eq. (12).

$${argmin}_{{q}_{1}^{*} , {q}_{3}^{*}} \frac{1}{2} {T}^{T} W ( d ) T \\ where T = ⌊ {S}_{c} ( d ) {\tilde{θ}}_{c} - ( {S}_{e} ( d ) {\tilde{θ}}_{e} ( {q}_{1} ) + {q}_{3} ( d ) ) ⌋ \\ and {argmin}_{x} f ( x ) ∈ \{ x \vert ∀ y : f ( y ) ≧ f ( x ) \} \qquad ( 12 )$$

denotes the value of x that minimizes f(x). At insertion depth d, Sₑ(d)=[0_{L-d} I_{d}] where I_{d} represents the inserted part of electrode array inside the scala tympani and 0_{L-d} is the un-inserted part of the electrode array. S_{c}(d)=[I_{d} 0_{L-d}] denotes the length from the entrance cₑₙₜ of the scala tympani to the point where the electrode array tip reaches cₜᵢₚ is d. W(d) is a weight matrix which specifies different weights to the steerable electrode array, from the tip to the base part. By varying these weights in the path planning, inventors can decide which portion of the electrode array simulates the curve of the scala tympani better. For any given insertion depth d, the optimal bending of the electrode array q₁* and the optimal robot base rotation q₃* are found. In this case, the angle differences between the inserted part of the electrode array and the scala tympani model are the smallest.

### Desired Position Determination and Inverse Kinematics of the Insertion Unit

For any given insertion depth d, when the optimal orientation is determined, the position of the electrode array with respect to the scala tympani is constrained by the entrance of the scala tympani cₑₙₜ. To achieve position optimality, in the present embodiment (FIG. 18) the offset t is given by

t(d,q₁*,q₃*)=cₑₙₜ−eₑₙₜ(d,q₁*,q₃*) (13)

Therefore, the determined desired result of the electrode array position and orientation is given by

pₑ*(s,q₁*,q₃*)=p_{c}(s−(L−d))+t(d,q₁*,q₃*) (14)

where p_{c}(s) represents the point of scala tympani at arc length s in {w}, pₑ*(s, q₁*, q₃*) represents the point of the electrode array at arc length s in {w}, and L−d≦s≦L. The determined desired result, according to the present embodiment, is shown in FIG. 19 where the inserted portion of the electrode approximates its corresponding curve of the scala tympani while respecting the constraint of the entrance point to the scala tympani. From the present determined desired results, FIG. 19 shows the position of the robot gripper, Eq. (15).

o_{g}*(q₁*,q₃*)=pₑ(L−d,q₁*,q₃*) (15)

The inverse kinematics of the robot gripper, as depicted in FIG. 19, can be solved to yield the desired results.

### Experimental Calibration and Simulation Results

In the experimental calibration process, inventors digitized 13 marked points (r=13) on the steerable electrode array and took a series of 12 images (z=12) to get the experimental data matrix Φ which is a 13 by 12 matrix. By solving Eq. (16), the solution of the calibration matrix is:

$$A = [ \begin{matrix}7.905 \times {10}^{- 6} & - 1.229 \times {10}^{- 5} & - 3.819 \times {10}^{- 4} & 4.754 \times {10}^{- 4} \\ - 1.162 \times {10}^{- 3} & 1.057 \times {10}^{- 2} & 5.942 \times {10}^{- 3} & - 2.576 \times {10}^{- 2} \\ 2.653 \times {10}^{- 2} & - 2.555 \times {10}^{- 1} & 2.532 \times {10}^{- 1} & 4.403 \times {10}^{- 1} \\ - 1.285 \times {10}^{- 1} & 1.160 & - 1.645 & - 1.839\end{matrix} ] \qquad ( 16 )$$

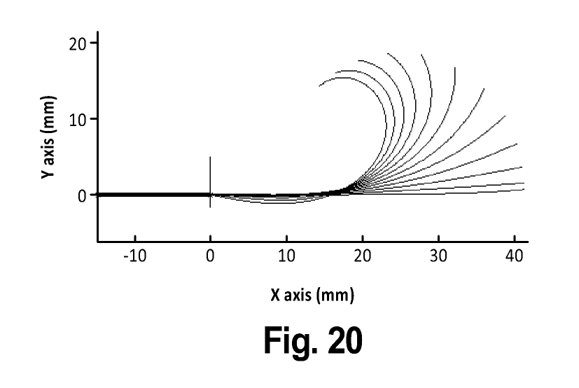

Before simulating the process of the electrode array insertion, inventors plotted different bent shapes of the electrode array with the same values of q₁ used in the calibration process. FIG. 20 provides simulation results of bent electrodes with the shapes of the electrode throughout its full range of motion. The shapes of the electrode array in FIG. 20 correspond well with the calibration images shown in FIG. 17. While the shape range simulated in FIG. 20 meets the criterion of the present embodiment, other criterion could be used. The calibration and simulation techniques disclosed herein can be modified and adapted by one of skill in the art.

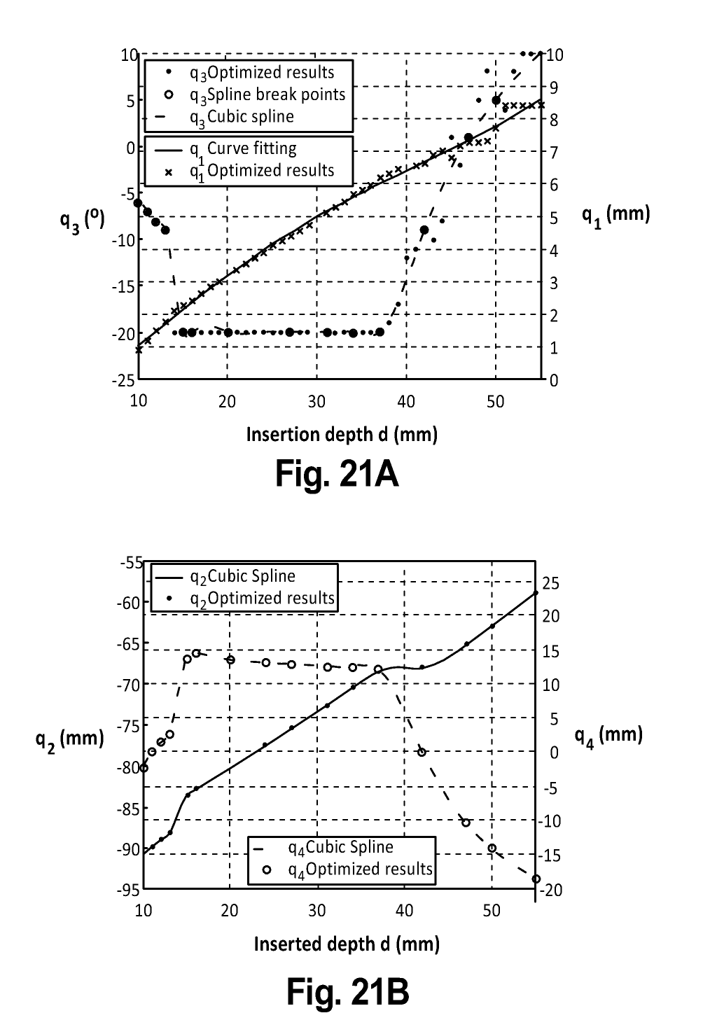

Given the calibration matrix A as in Eq. (16), the path planning determination was solved by applying the objective function, Eq. (12). Inventors started searching for the optimal value of the objective function from insertion depth d=10 mm to d=55 mm with increments of) 1 mm. Correspondingly, the range of rotation angle q₃ was restricted in (−20°,20°) with increments of 10. The pull of the actuation strand for optimal q₁ was calculated from 0 to 8.5 mm with a coarse increment of 0.7 mm. Once the most appropriate value was found, inventors searched for a more accurate value with fine increments ( 1/20 of coarse increments) within two nearby optimal values. The determined desired results for bending of the electrode array q₁* and the base rotation angle q₃* are shown in FIG. 21A, which illustrates the results for path planning given bending of electrode array q₁ and the electrode array base rotation q₃, in the present embodiment. The continuous solid line shows the results of a fourth-order polynomial fitting of the determined desired q₁* (discrete cross points). Eq. (17) gives the resulting polynomial with its coefficients. A single-parametric cubic spline (dashed line) was applied to approximate the determined desired q₃* (discrete dots). A number of u segments (u=15) were picked along the curve, and the segment break points are defined for insertion depth values given by d=[10, 11, 12, 13, 15, 16, 20, 24, 27, 31, 34, 37, 42, 47, 50, 55]. Each segment takes the form of Eq. (18).

For any spline segment j, the coefficients b_{j,i}(i=1, 2, 3, 4) are given by Eq. (19). Using the chord approximation, all the tangent vectors are solved by Eq. (20) where q₃′=[q_{3,1}′q_{3,2}′, . . . , q_{3,u}′]^{T} and matrices M and R are given by Eq. (21) and Eq. (22)

$$\begin{matrix} {q}_{1}^{*} ( d ) = {a}_{4} \times {d}^{4} + {a}_{3} \times {d}^{3} + {a}_{2} \times {d}^{2} + {a}_{1} \times d + {a}_{0} \\ {a}_{4} = 9.6345 \times {10}^{- 7} , {a}_{3} = - 8.7915 \times {10}^{- 5} , \\ {a}_{2} = 8.7275 \times {10}^{- 4} , {a}_{1} = 2.3942 , {a}_{0} = - 1.3966 & ( 17 ) \\ {q}_{3} ( d ) = {\sum }_{i = 1}^{4} {b}_{j , i} {d}^{i - 1} , {d}_{j} < d < {d}_{j + 1} for segment j , j = 1 , 2 , … , u & ( 18 ) \\ {b}_{j} = [ \begin{matrix}{b}_{j , 1} \\ {b}_{j , 2} \\ {b}_{j , 3} \\ {b}_{j , 4}\end{matrix} ] = [ \begin{matrix}1 & 0 & 0 & 0 \\ 0 & 1 & 0 & 0 \\ - 3 / {d}_{j + 1}^{2} & - 2 / {d}_{j + 1} & 3 / {d}_{j + 1}^{2} & - 1 / {d}_{j + 1} \\ 2 / {d}_{j + 1}^{2} & 1 / {d}_{j + 1}^{2} & - 2 / {d}_{j + 1}^{3} & 1 / {d}_{j + 1}^{2}\end{matrix} ] [ \begin{matrix}{q}_{3 , j} \\ {q}_{3 , j}^{′} \\ {q}_{3 , j + 1} \\ {q}_{3 , j + 1}^{′}\end{matrix} ] & ( 19 ) \\ {q}_{3}^{′} = {M}^{- 1} R & ( 20 ) \\ M = \{ \begin{matrix}{m}_{j - 1 , j} = {d}_{j + 1} , {m}_{j , j} = 2 \times ( {d}_{j} + {d}_{j + 1} ) , {m}_{j + 1 , j} = {d}_{j} & for 2 ≦ j ≦ u - 1 \\ {m}_{1 , 1} = {m}_{u , u} = 1 & \\ {m}_{i , j} = 0 & elsewhere\end{matrix} & ( 21 ) \\ R = \{ \begin{matrix}{r}_{j , 1} = \frac{3}{{d}_{j} {d}_{j + 1}} ( {d}_{j}^{2} ( {d}_{j + 1} - {d}_{j} ) + {d}_{j + 1}^{2} ( {d}_{j} - {d}_{j - 1} ) ) & for 2 ≦ j ≦ u - 1 \\ {r}_{j , 1} = {q}_{3 , j}^{′} & for j = 1 or j = u\end{matrix} & ( 22 )\end{matrix}$$

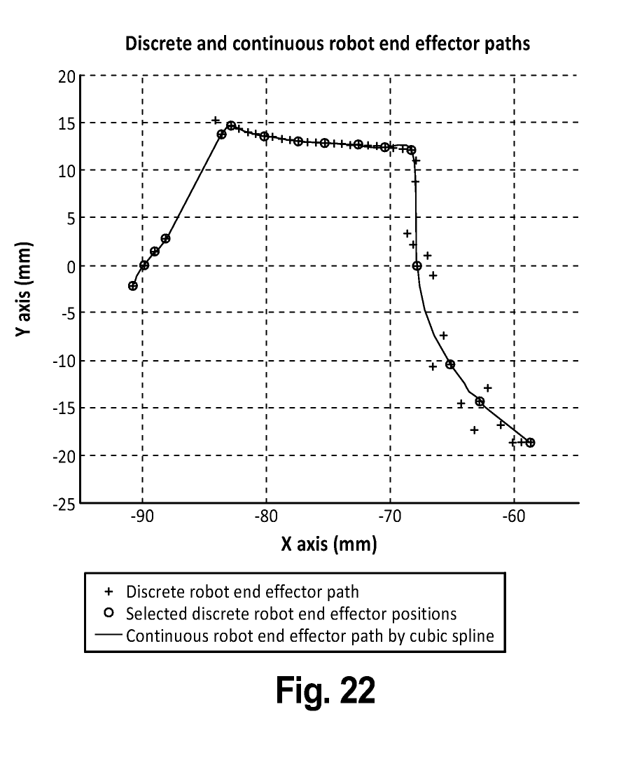

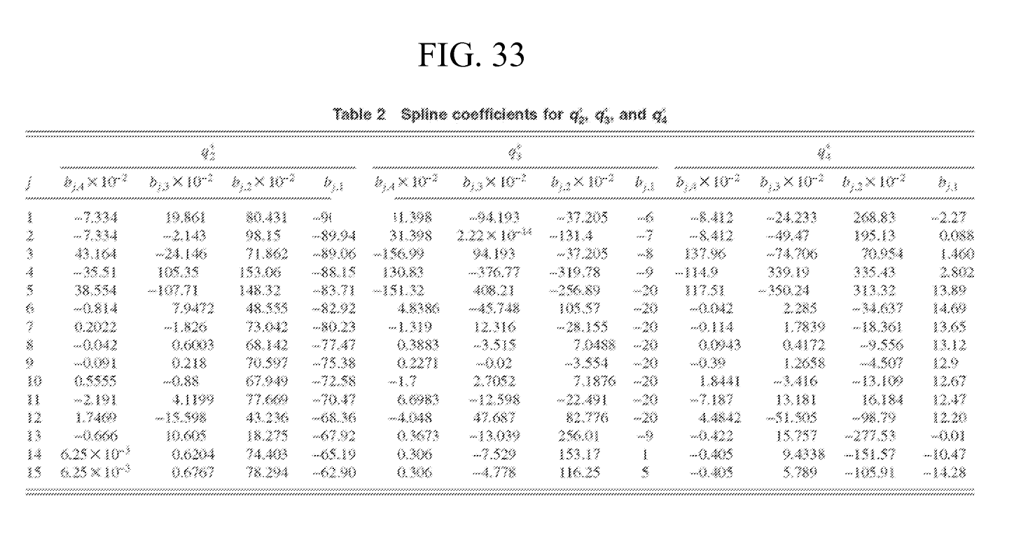

By solving the inverse kinematics using the determined results, discrete end effector positions (discrete dots) are plotted in FIG. 22, which illustrates spline results of the end effector path for the under-actuated robot. It shows the desired motion of the gripper for optimal insertion of the steerable electrode array. FIG. 21B shows the determined desired values for q₂* (discrete dots) and q⁴* (discrete circles) based on Eqs. (18)-(20). FIG. 21B depicts the results for path planning in which prismatic joint q₂ and prismatic joint q₄ are controlled. These values give the optimal translation of the gripper that match the path in FIG. 22. The paths for the prismatic joints after cubic spline interpolation (solid and dashed lines) are also shown in FIG. 21B. Details regarding the spline coefficients used are summarized in FIG. 33 (Table 2).

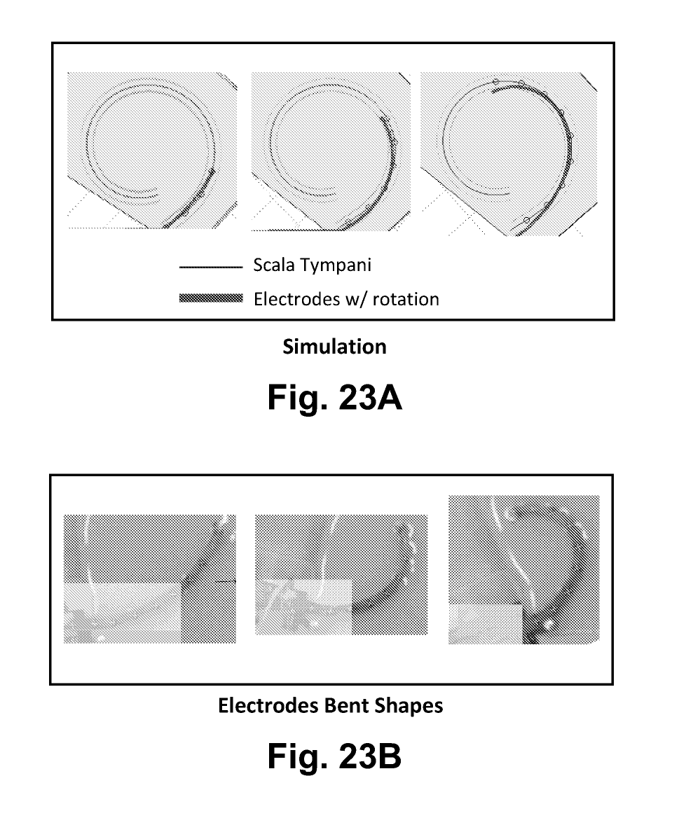

FIG. 23 demonstrates the effectiveness of the insertion path planning based on the results disclosed above. FIG. 23A provides the simulation results for an insertion based on the calibrated model of the electrode shown in FIG. 23B. Comparison of FIGS. 23A and B shows that the optimal position and orientation of the electrode was successfully found in order to optimally fit the shape of the scala tympani.

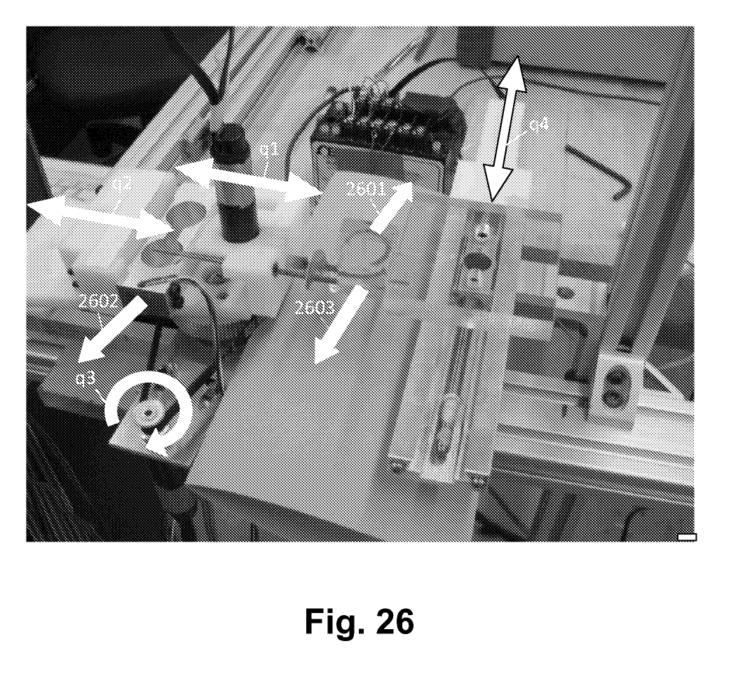

As noted above, FIG. 21 illustrates the optimal rotation and translation of the electrode base, in accordance with the present embodiment. The present set of rotation and translation parameters are enacted by employing a four DoF electrode insertion setup as shown in FIG. 26. Inventors compare this 4DoF setup with a simpler 2DoF setup, in which the orientation of the electrode base is constant and the translation of the electrode base is only in the insertion direction while the electrode is steerable.

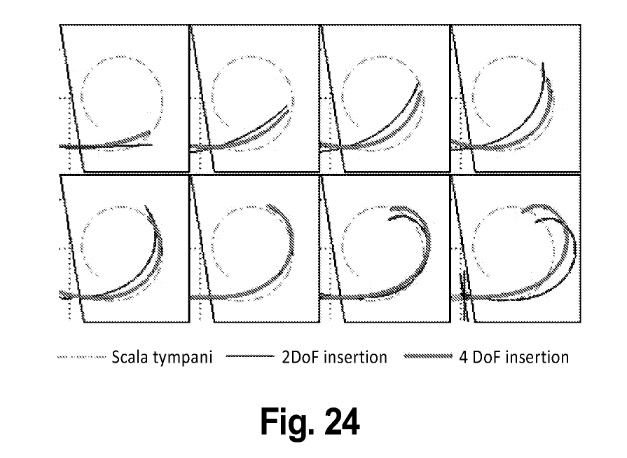

A simulation of the insertion process for each of the 2DoF and the 4DoF embodiments is shown in FIG. 24. The figure clearly shows that the 4DoF system, according to the present embodiment, provides a better shape fit between the electrode and the scala tympani curve when compared to the 2DoF system. To quantify the shape difference between the bent electrode array and the scala tympani curve, the simulated average angle and distance variations are articulated in Eq. (23) and Eq. (24).

$$\begin{matrix}\overline{θ} = \frac{1}{d} {\int }_{0}^{d} \vert {θ}_{c} ( s ) - {θ}_{e} ( L - d + s ) \vert d s & ( 23 ) \\ \overline{p} = \frac{1}{d} {\int }_{0}^{d} \vert \vert {p}_{c} ( s ) - {p}_{e} ( L - d + s ) \vert \vert d s & ( 24 )\end{matrix}$$

The average angle and distance variations provide quantitative measures that describe the shape discrepancies between the bent electrode array and the scala tympani model. Small values of these average variations give a better shape fit between the electrode array and the scala tympani. Hence, the insertion force will be smaller due to the small shape discrepancies.

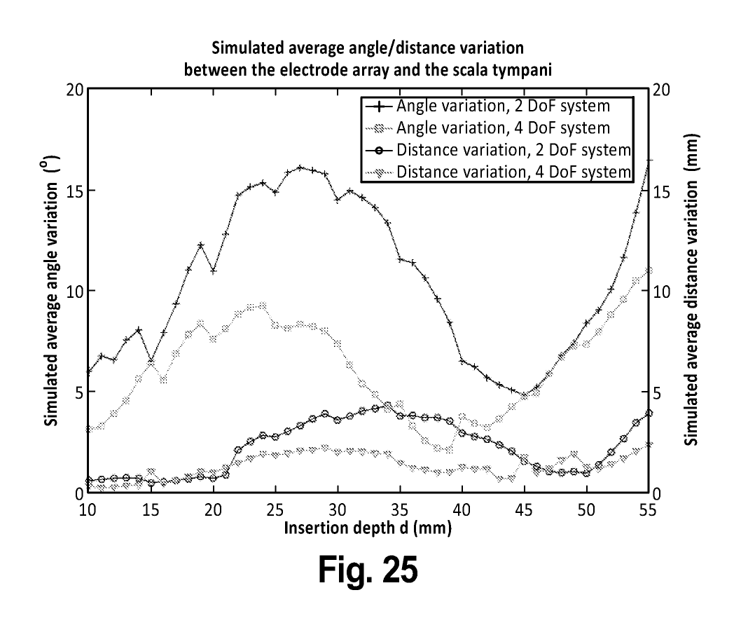

FIG. 25 provides a graph of quantitative measures the simulated average angle and distance variations between the electrode and the scala tympani for each of the 2DoF and 4DoF systems. As shown in the graph, during the insertion process, the 4DoF system retains a smaller angle and distance variations than the 2DoF system. These results agree with those results qualitatively depicted in FIG. 24. The agreement between the qualitative and quantitative findings provides justification for using a 4DoF system in which the steerable tip of the electrode is controlled as well as the 3DoF position and orientation of its base.

### Experimental Setup and Results

FIG. 26, introduced above, shows the experimental setup used to validate the insertion path planning FIG. 26 shows a steerable electrode array 2601 with a single axis force sensor 2602 and a three-dimensional scala tympani model 2603. Each of the four degrees of freedom, q₁, q₂, q₃, and q₄ are indicated with corresponding arrows. It uses scaled up (3:1) models of the scala tympani. The setup using a 3D model of the scala tympani is shown. The plane in which the electrode bends is tilted at a certain angle to match the helix angle of the scala tympani model. A scaled up (3:1) model of a typical electrode array was fabricated using silicone rubber. An AG NTEP 5000d single axis force sensor was used in the present setup to measure the axial insertion force of the electrode array and it was capable of detecting ±0.1 g force using a 13 bits A/D acquisition card. The robot position control was achieved using Linux Real Time Application Interface (RTAI) with a closed loop rate of 1 KHz. Other experimental setups are envisioned and may be appropriate for certain circumstances.

In the present experiment, each setup, insertions using 1DoF (non-steerable electrode array), 2DoF and 4DoF experimental systems (using steerable electrode array) were carried out. For 1DoF insertions, only the actuation unit q₂* is activated. 2DoF insertions use both joints q₁ and q₂ so that the electrode array is steered according to the pre-defined path. For the 4DoF system, all joints q₁, q₂, q₃, and q₄ are actuated.

Those experiments using ISFF used the linear proportional control law as given in Eq. K>0 f (25). The proportional gain K_{f}≧0 was related to the magnitude of the insertion force fᵢₙₛ. K_{f} approaches zero as the insertion force fᵢₙₛ increases up to a predetermined value fₘₐₓ. The values used for Vₘₐₓ and Vₘᵢₙ were determined based on clinical observations and previous works.

$${V}_{ins} = {V}_{min} + {K}_{f} ( {V}_{max} - {V}_{min} ) \\ where {K}_{f} = \{ \begin{matrix}( {f}_{max} - {f}_{ins} ) / {f}_{max} & {f}_{ins} ≦ {f}_{max} \\ 0 & {f}_{ins} > {f}_{max}\end{matrix} \} . \qquad ( 25 )$$

The parameters used are fₘₐₓ=50 g, Vₘₐₓ=2 mm/s and Vₘᵢₙ=0.22 mm/s

Table 1 presents the experimental conditions tested using different experimental setups. In order to validate repeatability, the experiments were repeated three times for each experimental setup and insertion condition. In all cases, the same prototype electrode array,

TABLE 1
Experimental conditions

| | Insertion | | | |
|---|---|---|---|---|
| | speed | | | |
| | force | 2D scala | 3D scala | |
| | feedback | tympani | tympani | FIG. |
| 1DoF | | ♦ | | 30A |
| setup | ♦ | ♦ | | 30C |
| | ♦ | | ♦ | 21 |
| 2DoF | | ♦ | | 30B |
| setup | ♦ | ♦ | | 30D |
| | ♦ | | ♦ | 31 |
| 4DoF | | ♦ | | 33B |
| setup | | | ♦ | 33A |

calibrated in FIG. 7 was used when comparing non-steerable electrode array insertions with steerable insertions.

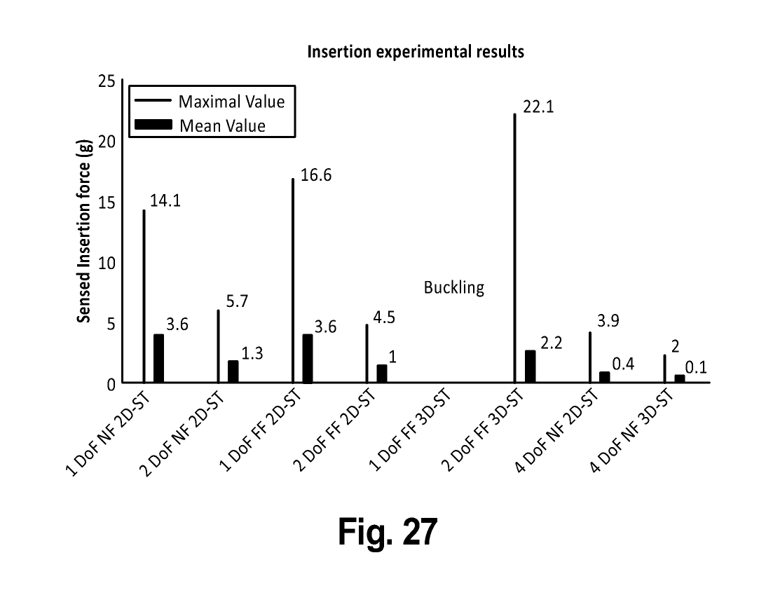

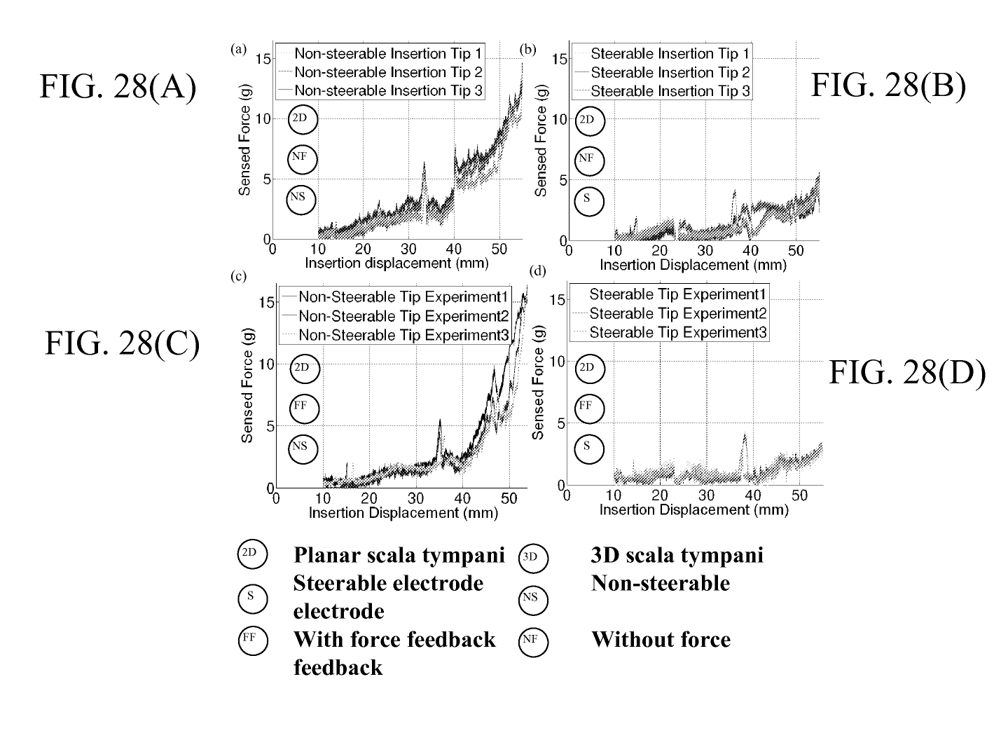

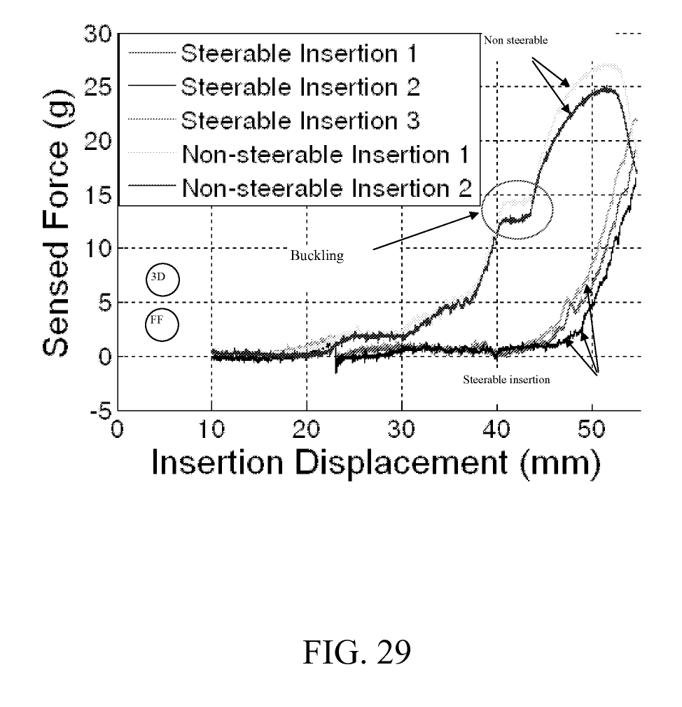

FIG. 27 shows the insertion experimental results for average and maximal sensed forces for each experimental condition given in Table 1. Inventors note that those insertions using the non-steerable electrode array into 3D scala tympani with ISFF did not achieve full insertion as a result of buckling. This result is also depicted in FIG. 28 and FIG. 29A, discussed below.

### Analysis of the Results of One and Two DoF Insertions

FIG. 28 shows the experimental results of insertions into planar scalar tympani model using a two DoF system. FIG. 28A illustrates sensed forces as a function of insertion displacement for a non-steerable electrode array without ISFF. FIG. 28B illustrates sensed forces as a function of insertion displacement for a steerable electrode array without ISFF. FIG. 28C illustrates sensed forces as a function of insertion depth for a non-steerable electrode with ISFF and FIG. 28D illustrates the case of a steerable electrode with ISFF. Comparing FIG. 28B with FIG. 28A the reduction of insertion force is obvious by using the steerable electrode array. The maximal insertion force is reduced by 59.6%. Comparing FIG. 28D with FIG. 28C, using the steerable electrode reduces the maximal insertion force by 72.3%. All the groups in FIG. 28 use the planar scala tympani model and the force impulse around d=35 mm is caused by the first contact between the tip of the electrode array and the outer wall of scala tympani model. The same impulse force was observed in FIG. 28B.

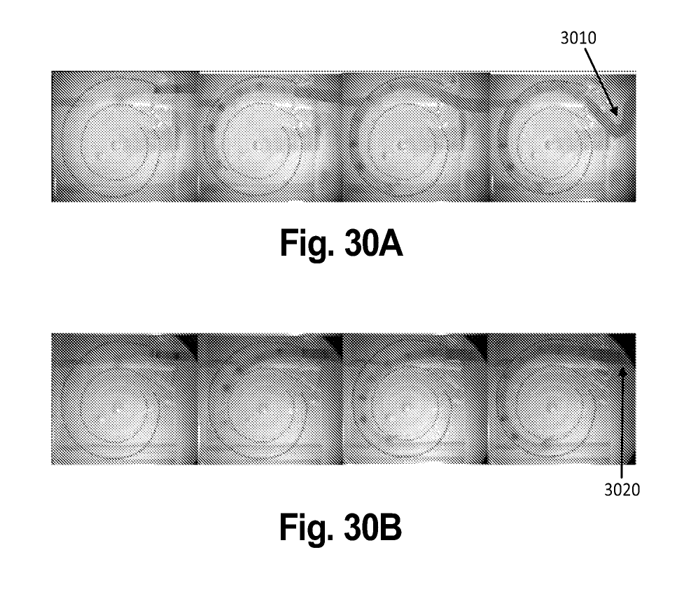

FIG. 29 shows the insertion forces during insertions of the same non-steerable electrode array into the 3D scala tympani model. FIG. 29 plots insertion forces found during experiments using a two DoF system with ISFF for both non-steerable and steerable electrode arrays. For the non-steerable electrode array, the insertion force is large enough to generate buckling of the electrode array. The encircled portion of the force readings, highlighted in FIG. 29, shows a sudden decrease in the electrode stiffness due to buckling. This phenomenon is confirmed by the insertion video snapshots in FIG. 30 A-B. FIG. 30A depicts photographs taken during test insertions using a non-steerable two DoF system and a three dimensional scala tympani model, with ISFF, in which buckling 3010 occurred. Using the steerable electrode array, the insertion can be achieved without buckling in region 3020, as shown in FIG. 29B.

In FIG. 29 the insertion force for the steerable electrode array increases quickly for insertions deeper than 45 mm because the geometric constraints of the scala tympani model do not allow further insertion of the electrode array. The planar scala tympani model does not present this problem because the model has a uniform cross section along the backbone curve of the scala tympani.

Due to the transparent property of the 3D scala tympani model, the boundaries of the scala tympani chamber less visible in the images in FIG. 30. Hence, inventors emphasize the chamber boundaries by digitally highlighting the two boundary curves in FIG. 30.

FIG. 27 compares the insertion forces for non-steerable insertions with and without insertion speed force feedback (ISFF)(FIG. 28C and FIG. 28A). Using the control law of Eq. (25) caused a 17.7% increase in the maximal insertion force while maintaining the same average insertion force. For 2DoF steerable insertions (FIG. 28D and FIG. 28B), using ISFF according to Eq. (25) reduced the insertion forces by 21.1% and 23.1% in maximal and average insertion forces, respectively. These results pointed to the desirability of further research to determine the optimal ISFF control law that replaces Eq. (25) and provides consistent results for steerable and non-steerable electrode arrays. This control law depends on determining the correct friction model that takes into account viscous, stiction, and hydrodynamic effects. Modeling the effect of friction during insertion and its impact on insertion forces for the electrode arrays is detailed below.

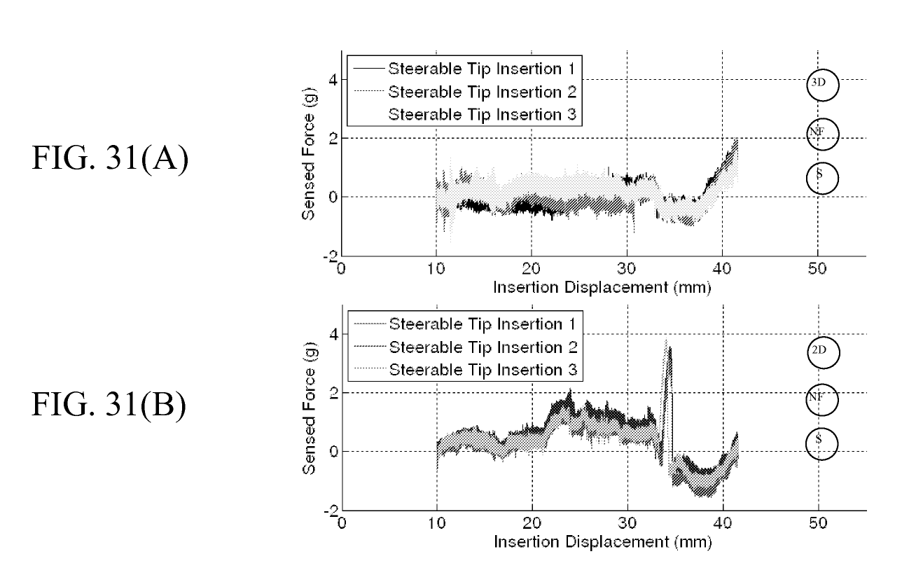

A set of experiments was carried out using the 4DoF insertion system, FIG. 25 using the same steerable electrode array without ISFF. FIG. 31 shows the results of inserting the steerable electrode array into the planar scala tympani model and the 3D scala tympani model. FIG. 31A depicts the sensed force measured for the steerable electrode array insertions using a 4 DoF system without ISFF and a planar (2D) scala tympani model. FIG. 31B depicts the sensed force measured for the steerable electrode array insertions using a 4DoF system without ISS and a 3D scala tympani model. In both cases, the insertion forces are only 3.9 grams and 2.0 grams because of using the steerable electrode array of the present embodiments.

For 4DoF insertion system, the insertion is achieved up to 42 mm. Comparing FIG. 31B with FIG. 28B, the maximal insertion force for the present embodiments was reduced from 4.8 grams to 3.9 grams, 18.8% reduction was observed. Similarly, comparing FIG. 31A with FIG. 29A, the maximal insertion forces up to 42 mm for the present embodiments are comparable. However, inventors expect that as the insertions go deeper, using 4DoF system will further decrease the insertion force compared with 2DoF system.

In the case where steerable electrode array was used (FIGS. 28, 30D, 31 and 33) negative forces happened when the steerable electrode array was steering and hugs the inner wall of the scala tympani model.

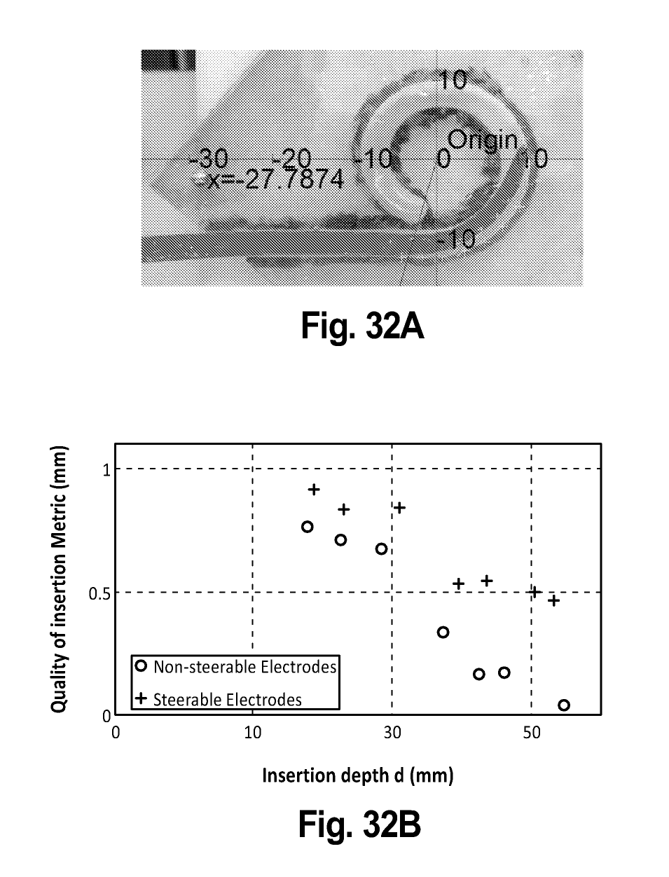

For the experiments with 2DoF system and the planar scala tympani model, FIG. 28, inventors digitized the edge of the scala tympani and the steerable electrode array for both non-steerable and steerable electrode arrays. FIG. 32A shows a digitization of the average distance measures during insertion. FIG. 32B illustrates a quality of insertion metric for each of non-steerable and steerable electrodes, as discussed below. In this example, 7 pictures are digitized for each set.

The actual average distance between the electrode array and the scala tympani model can be defined as the quality of insertion metric, Eq. (26).

$$\overline{p} = \frac{1}{d} {\int }_{0}^{d} \vert \vert {r}_{c} ( s ) - {r}_{e} ( L - d + s ) \vert \vert d s \qquad ( 26 )$$

where r_{c} (s) represents the actual point of scala tympani at arc length s in {w}, rₑ (s) represents the actual point of the electrode array at arc length s in {w}.

As opposed to the simulated average angle and distance variations, which are defined in Eq. (23) and Eq. (24), the quality of insertion metric describes the actual average distance between the electrode array and the scala tympani model during the insertion process. The quality of insertion metric may be calculated and compared in FIG. 32B for both the non-steerable and steerable electrode array. In both steerable and non-steerable insertions, the actual average distance decreases as the insertion depth is increasing. However, the decrease rate is slower for the steerable electrode array than for the non-steerable electrode array implying the steerable electrode array provides bigger gap between the electrode array and the scala tympani during the insertion process. Hence it explains the reduced insertion force when using the steerable electrode array.

As noted above, during certain experiments, it has been found that insertion forces not only depend upon the shape discrepancy between the scala tympani and the inserted electrode array, but also on the insertion speed. Thus, additional embodiments of robot-assisted cochlear implant surgery implement methods and implant parameters that account for friction considerations. Friction models and parameter identification enables insertion of cochlear implant electrode arrays, wherein the insertions forces are further reduced. The friction model discussed below describes the whole insertion process and investigates the relationship between the insertion speed and the insertion force. Experimental and statistical results show the effectiveness of the model. Applying the friction model generates safety insertion force boundaries for future insertions and gives the optimal insertion speed. It also provides predictive force information for insertion speed feedback control law design which may be applied to robot-assisted cochlear implant surgeries.

Model and Parameter Identification of Friction during Robotic Insertion

Inside the cochlea, referring again to FIG. 2B, the electrode array is inserted into the scala tympani, which is the bottom channel of the inner ear, filled with bodily liquid. Experimental and clinical trials have shown various traumas, including punching through the delicate basilar membrane or damaging the cochlea bones, during the insertion process. The traumatic rate of manual insertions is reported at approximately 30% and above, according to the literature and it is expected that large insertion forces will cause serious trauma. As disclosed above, the relationship between insertion angle (which in turn depends upon insertion depth) and the insertion force is a crucial factor taken into consideration, when designing feedback control law for robot assisted cochlear implant surgery. The insertion force prediction can be further refined by providing an explicit model that relates insertion force to insertion speed. A definitive analysis of the insertion speed complements the analysis of friction in cochlear implant surgery, contact pressure distribution, insertion force profiles, etc. A statistical analysis to generate safe electrode array insertion force boundaries is the focus of the following discussion.

The friction coefficient between the electrode array and the endosteum lining has been computed using a band break model (i.e. friction coefficients for standard straight electrode array with and without lubricants such as glycerin). Standard Finite Element Analysis (FEA) methods have been used to analyze the insertion of a flexible beam into a straight hole with surfaces contacting. FEA methods have been used to calculate the contact pressure between the electrode array and the scala tympani external wall. Inventors have found that the relationship between the contact pressure and a sensed insertion force and insertion speed have been inadequately addressed in the literature. Inventors have determined that models based on a quasi-static equilibrium assumption insufficiently capture the effects of friction.

The present embodiments focus on a physical model which may be used to calculate the total insertion force at any given insertion angle (and depth). Also, the relationship between insertion speed and insertion force is determined. Statistical results show the effectiveness of the model and give the safety force boundaries for electrode array insertions. All of these help to design an insertion speed feedback control law for a customized robot which will be used for cochlear implant surgery. The discussion below shows the system description, the insertion force model, the simulation results and the experimental results.

### Surgical Needs and System Description

In a typical clinical setup for cochlear implant surgery, two surgeons operate under a microscope with their electrode array insertion tools. Insertion tools may include, for example, standard tweezers, rat claws, advance off-stylet tool, etc. With different tools, standard insertion technique, advance off-stylet technique, or partial withdraw technique could be used. However, none of these tools or techniques provides a direct force measure or feedback to the surgeons.

Different electrode array designs have also been proposed and applied. The most common electrode array is a standard straight electrode array which has a tapered shape from the bottom to the tip while some products may use a softer tip. Some other electrode arrays are pre-coiled with a platinum sheath in the middle. Once the sheath is pulled out, the electrode array coils into a curve that is similar to the scala tympani. These passive flexible electrode arrays are usually very small (less than 1 mm in diameter), flimsy and buckle easily during insertions.

With limited force information collected from the tools and inadequate control over the shape of the electrode array, the insertion is very difficult to perform manually. Although fluoroscopy imaging helps surgeon see how the electrode array is inserted, the application of such technique during insertions is very rare. In order to achieve a smaller insertion force, a steerable electrode array was designed. Preliminary research has indicated that robot assisted cochlear implant, according to the present embodiments, can reduce the insertion force significantly.

In certain embodiments, a manual electrode array insertion process can be finished by a robot that is manipulated by a surgeon. Inventors have identified the importance of providing an appropriate insertion speed feedback control law for robotic control, that helps achieve optimal insertion with reduced insertion force. This control law design is provided based upon a physical model that describes the insertion force versus insertion angle (which in turn depends upon insertion depth) and insertion speed. Inventors propose such a control law while retaining a safety boundary of insertion forces enabling the surgeons to intervene if the force exceeds the limit.

### System Description

The anatomical structure of the cochlea is a 3D spiral curve. Incorporated reference Cohen et al. used a statistical method in characterizing the geometric dimension of planar scala tympani. The backbone curve of the scala tympani is expressed in Eq. (25), where r, z, and θ are the cylindrical coordinates of this curve (r is the radial distance to the curve, z is the height, and θ is the angle). The values of the constants a, c, b, d, θ₀, p are based on known conventions in the literature.

$$R = \{ \begin{matrix}c ( 1 - d log ( θ - {θ}_{0} ) ) & θ < 100 ° \\ a {e}^{- b θ} & θ ≧ 100 °\end{matrix} \} \\ z = p ( θ - {θ}_{0} ) , θ ∈ [ 10.3 ° , 910 ° ] \qquad ( 25 )$$

Based on this present model, Cochlea Inc. created planar scala tympani models that are used for training surgeons. Inventors have conducted experiments on one of these models to measure insertion forces. Because the model is transparent from the top, it provides good conditions for imaging afterwards.

The commercial external wall (straight) electrode array used in these experiments is from MedEl Corp. but other suitable apparatus may also be used. Its fully inserted length is about 26 mm long with a 1.2 mm diameter bottom tapered into a 0.6 mm diameter tip. The size is relatively large compared to some of other commercial products. A total number of 11 platinum bands are distributed evenly from the tip of the electrode array. In certain embodiments, inventors use these straight electrode arrays. Inventors have found this embodiment to be desirable in certain cases, because when inserted, due to bending of the electrode arrays, the straight electrode arrays provide approximately full contact between the electrode arrays and the scala tympani external wall. This facilitates the calculation for friction.

### Modeling

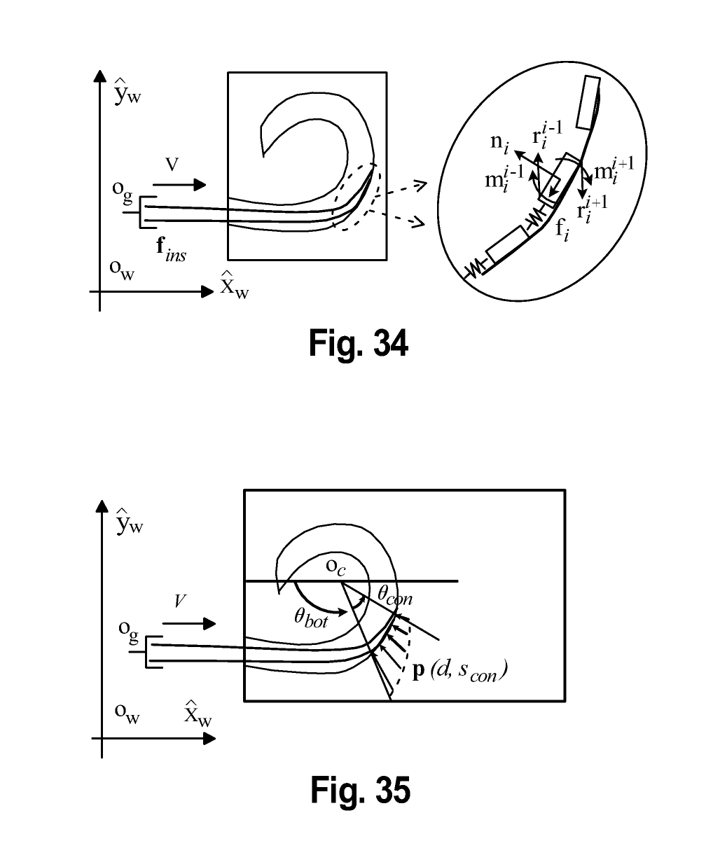

The electrode array is essentially a flexible beam. It is inserted into a rigid planar scala tympani model fixed on a platform. FIG. 34 illustrates a planar (2D) static modeling of an electrode array where the flexible beam is divided into multiple rigid elements. In the present model, the multiple rigid elements are connected through spring joints which transmit compression and torsion between rigid elements. This model is expressed below in Eq. 26.

$$\{ \begin{matrix}{\sum }_{i = 1}^{n} ( {n}_{i} + {f}_{i} + {r}_{i}^{i - 1} + {r}_{i}^{i + 1} ) + {f}_{ins} = 0 \\ {\sum }_{i = 1}^{n} ( {m}_{i}^{i - 1} + {m}_{i}^{i + 1} ) = 0\end{matrix} \qquad ( 26 )$$

Static equilibrium of element i is shown in Eq. 26, where mᵢ^{j}(j=i−1,+1) includes torque generated from torsional spring and the connecting element. Since the relationship of internal forces generally holds, Eq. 27. The combined force at the end of the electrode array is given by Eq. 28.

$$\begin{matrix}{r}_{i}^{i - 1} + {r}_{i - 1}^{i} = 0 & ( 27 ) \\ {\sum }_{i = 1}^{n} ( {n}_{i} + {f}_{i} ) + {f}_{ins} = 0 & ( 28 )\end{matrix}$$

Using a single degree of freedom force sensor embodiment, the only force sensed is along the insertion direction which is \hat{x}_{w}. Therefore, the amplitude of the sensed force is given by Eq. 29.

Fᵢₙₛ_{_{—}}ₓ=fᵢₙₛ·x_{w} (29)

To calculate the normal force nᵢ and local friction force fᵢ, a contact pressure distribution was assumed between the inserted electrode array and the scala tympani model. This pressure distribution is solved and follows an approximately linear distribution from the electrode array tip along the contacted portion. This is schematically represented in FIG. 35, which summarizes the electrode array contact pressure distribution.

The assumed contact pressure distribution is shown in Eq. 30.

$$\tilde{p} ( d , {s}_{con} ) = {p}_{tip} ( d ) + \frac{{s}_{con}}{{l}_{con}} ( {p}_{bot} ( d ) - {p}_{tip} ( d ) ) \qquad ( 30 )$$

where o_{c} is the center of the scala tympani model. l_{con} represents the contacted arc length. s_{con}=0 is the tip of the electrode array and s_{con}=l_{con} is the point where the electrode array starts leaving the external wall of the scala tympani. Contact start angle θ_{bot}, is the angle where l_{con} locates and θ_{con} is the contact angle. This pressure distribution is also a function of electrode array insertion depth d.

The Stribeck friction model was used. A common nonlinear friction model is given by Eq. 31,

$$f ( v ) = {f}_{c} + ( {f}_{s} - {f}_{c} ) {e}^{- {\vert v / {v}_{s} \vert}^{{δ}_{s}}} + {f}_{v} v \qquad ( 31 )$$

where fₛ=μₛN and f_{c}=μN are static and kinetic frictions. vₛ is Stribeck velocity and δₛ is a known constant. fᵥv represents the viscous friction which is negligible in this case.

Because the sensed insertion force Fᵢₙₛ_{_{—}}ₓ is a function of friction as shown in Eqs. 28 and 29, it is also understood to be a function of insertion speed v. By introducing Eq. 31 into the model, insertion speed is considered in the sensed force and may be implemented in the simulation and validated in experiments summarized below.

### Simulation Results

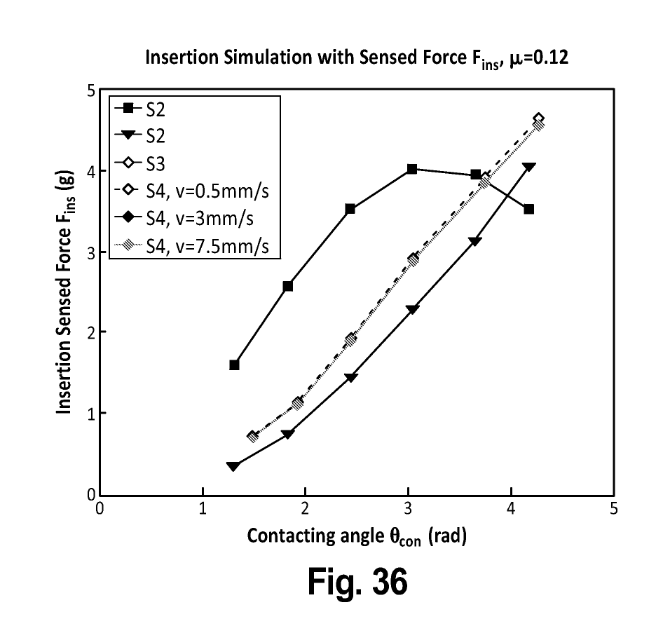

A two dimensional (2D) insertion simulation was conducted to calculate the insertion force Fᵢₙₛ_{_{—}}ₓ along the insertion direction based on given information. The kinetic friction coefficient between the electrode array and the Teflon scala tympani model was 0.12 obtained and a value of 0.18 was used for the kinetic friction coefficient. Four different scenarios have been considered and simulated using the values in Table 3 (below). Results of these simulations are shown in FIG. 36 and Table 4 (below). Specifically, FIG. 36 depicts an insertion simulation with selected sensed force over a range of contacting angles. Simulation 51 shows a fixed pressure range that is linearly distributed over the contacting area. The linear contract pressure distribution is shown as Eq. 30. As the contact angle θ_{con} increases, the contact pressure attenuates at any given position. Simulation S2 assumes a constant contact pressure at the tip and a linear increase in the pressure p_{bot} at the bottom. In Simulation S3, both pₜᵢₚ and p_{bot} change during insertion. The contact start angle θ_{bot} in FIG. 35 decreases, meaning that during insertions, the contact start point s_{con}=l_{con} moves backwards while the electrode array tip moves forward, resulting in a bi-directional expansion of contact between the electrode array and the whole scala tympani external wall. The values of the contact start angle in simulation S3 may be statistically obtained from preliminary experiments. Simulation S4 implement the Stribeck friction model in sensed insertion force Fᵢₙ_{_{—}}ₓ while keeping other parameters the same as S3.

FIG. 36 shows the simulations of the simulation results using MATLAB. S1 plot shows a decreasing insertion force as the insertion goes deeper. S2 to S4 show similar increasing sensed forces. In the present simulation, inventors observe a slight decrease in S4 compared to S3. Table 4 summarizes the simulation results of S4 based model. The present simulation indicates that in certain embodiments, the insertion force decreases when insertion speed increases.

TABLE 3
SIMULATION CONDITIONS

| Trial | Conditions | FIG. |
|---|---|---|
| 1 | θ_{bot} = 1.40 rad, pₜᵢₚ = 0.1 MPa, p_{bot} = 0.15 MPa | FIG. 36-S1 |
| 2 | θ_{bot} = 1.40 rad, pₜᵢₚ = 0.1 MPa, | FIG. 36-S2 |
| | p_{bot} increases linearly within the range [0.04, | |
| | 0.21 MPa | |
| 3 | pₜᵢₚ = [0.03, 0.04, 0.06, 0.08, 0.09, 0.1] MPa | FIG. 36-S3 |
| | p_{bot} = [0.04, 0.05, 0.07, 0.01, 0.15, 0.2] MPa | |
| | θ_{bot} = [1.83, 1.66, 1.48, 1.31, 1.13, 0.96] rad | |
| 4 | Same as 3 and with Stribeck friction model. | FIG. 36-S4 |

TABLE 4
SPEED EFFECT FOR SIMULATION S4

| | Simulated Sensed Force (grams) | | |
|---|---|---|---|
| θ_{con} (rad) | ν = 0.5 mm/s | ν = 3 mm/s | ν = 7.5 mm/s |
| 1.4835 | 0.7048 | 0.6918 | 0.6906 |
| 1.9199 | 1.1185 | 1.0994 | 1.0977 |
| 2.4435 | 1.9135 | 1.8861 | 1.8837 |
| 3.0543 | 2.9039 | 2.8691 | 2.8660 |
| 3.7525 | 3.8943 | 3.8454 | 3.8411 |
| 4.2761 | 4.6264 | 4.5510 | 4.5444 |

The simulation results depicted in FIG. 36 and Table 4 suggest that the Stribeck friction model coupled with a linear contact pressure distribution (Eq. 30) are suitable for modeling the observed behavior. The following sections employ the Stribeck friction model to describe experimental validations, describe the tuning of the Stribeck friction model, and disclose the validation of the values of θ_{con} as a function of insertion angle.

### Experimental Results

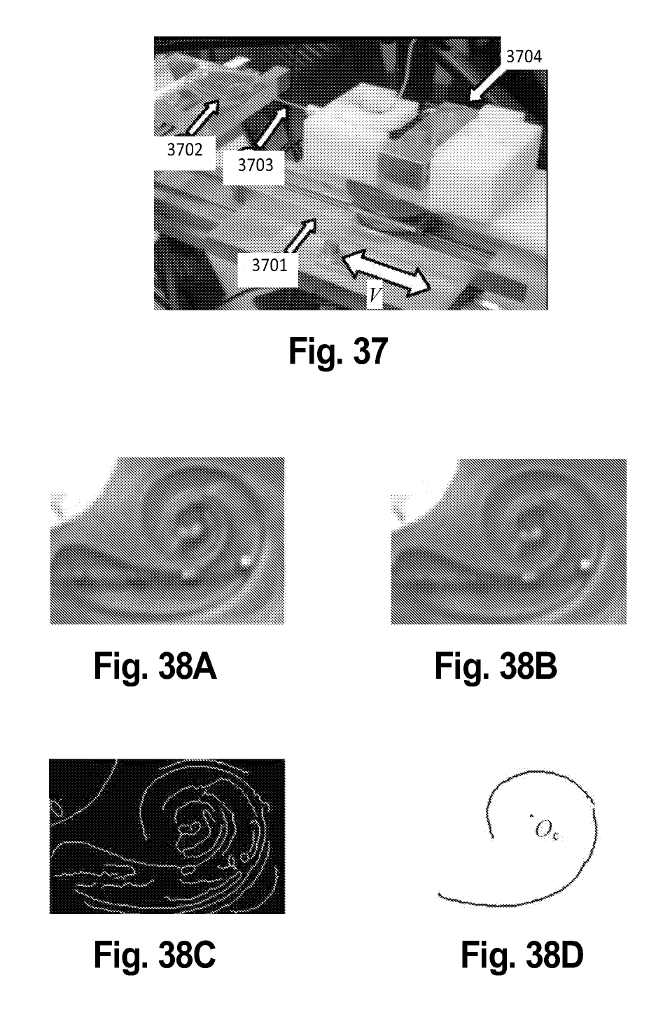

FIG. 37 shows the experimental setup with a planar 1:1 scala tympani model from Cochlear Inc. The system components illustrated in FIG. 37 include a single axis insertion robot 3701, a planar scala tympani model 3702, the electrode array 3703, and the single axis force 3704, according to certain embodiments. In the present embodiment, an AG NTEP 5000d single axis force sensor was used in the setup to measure the axial insertion force of the electrode array and it is capable of detecting ±0.1 g force using a 13 bit A/D acquisition card. The robot position control was achieved using Linux Real Time Application Interface (RTAI) with a closed loop control rate of 1 KHz. Constant insertion speed was achieved by offline path planning of the single axis robot.

When inserting the electrode array into the planar scala tympani model, the scala tympani channel was fully filled by glycerin solution, which is a common lubricant, to simulate in vivo conditions. Also, during insertions, an overhead video recorder was used to provide high resolution images. Other variations are envisioned to simulate the actual scala tympani channel environment.

In the present example, five groups of experiments were conducted with 5 different insertion speeds (0.5, 1.5, 3, 5, 7.5 mm/s). For the purposes of illustration, three groups are presented in greater detail below (0.5, 3, 7.5 mm/s). Yet other insertion speeds may be preferred in certain embodiments and under certain system parameters. Other insertion speeds were found to be were consistent with the three groups discussed in detail below. According to the present embodiment, the insertions in each group may be carried out up to 15 mm in depth. For each observed insertion speed v, each insertion was repeated 10 times to show repeatability and collect data for statistical analysis. Insertion process video clips were recorded for additional analysis.

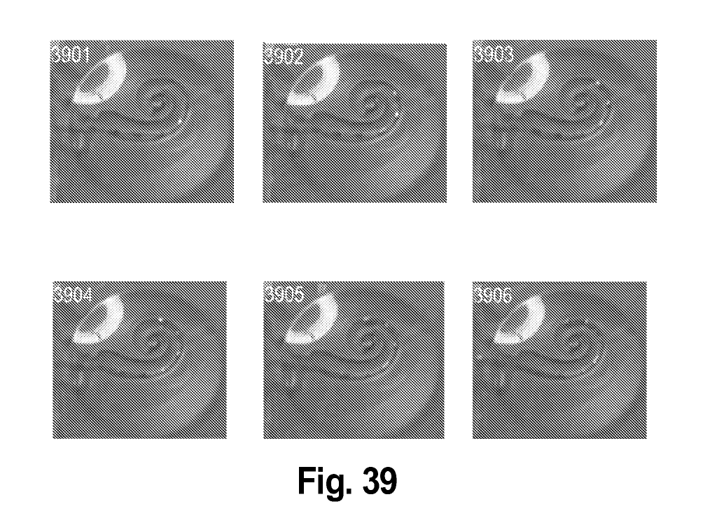

After the experiments, 25 images were captured from the video clip for each insertion process. Canny filters were applied and segmented images were used to register the center of the scala tympani model based on Eq. (25). FIG. 38 depicts segmentation insertion images include the raw image (FIG. 38A), the grey scaled image (FIG. 38B), the Canny filter image (FIG. 38C) and the resultant, image-based edge detection (FIG. 38D). Identification of the center allows the determination of the contact angle θ_{con} using the same method. FIG. 39 shows an insertion process that was digitized. The insertion process is depicted in six images (3901, 3902, 3903, 3904, 3905 and 3906) taken during six stages of the insertion process. Table 5 gives the angle values calculated from FIG. 39.

TABLE 5
SEGMENTATION RESULTS FOR INSERTION ANGLE (RAD)

| | FIG. | FIG. | FIG. | FIG. | FIG. | FIG. |
|---|---|---|---|---|---|---|
| | 39-1 | 39-2 | 39-3 | 39-4 | 39-5 | 39-6 |
| θ_{bot} | 1.68 | 1.41 | 1.26 | 0.94 | 0.77 | 0.84 |
| θ_{con} | 1.17 | 1.71 | 2.23 | 3.04 | 3.61 | 3.63 |
| θₜᵢₚ | 2.84 | 3.12 | 3.49 | 3.98 | 4.38 | 4.47 |

For the purposes of illustration the insertion force profiles were generated using saved data and plotted versus the contact angle. Further, the relationship between insertion force and insertion angle θₜᵢₚ was derived, since in the present model, θₜᵢₚ=θ_{bot}+θ_{con}.

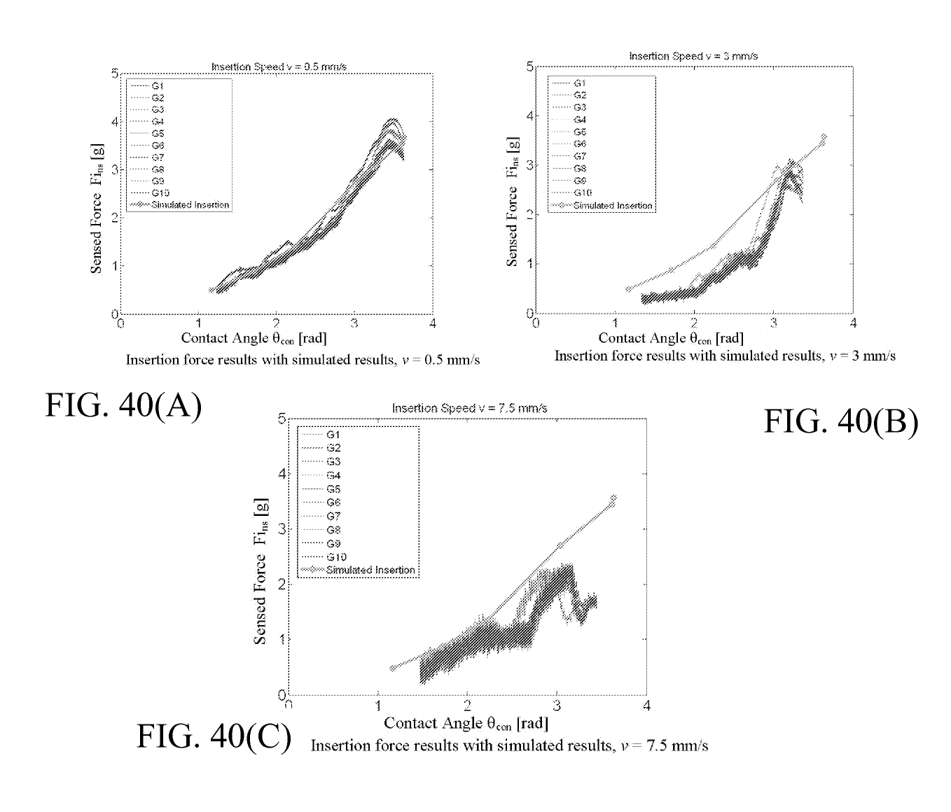

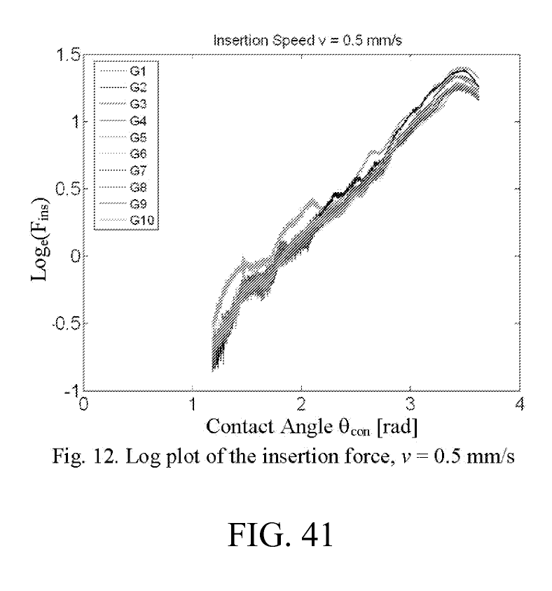

FIGS. 40A, B and C illustrate the experimental results with selected insertion speeds of 0.5, 3 and 7.5 mm/s, respectively. As shown in each plot, the insertion force profiles are highly repeatable. The corresponding simulation results are also superimposed on the figures, for the purposes of comparison.

In FIG. 40A, the simulated insertion matches with the experimental data very well. It is expected that one of skill in the art will be able to generate similar insertion force results and simulated results given appropriate parameter ranges. In each of FIG. 40B and FIG. 40C, the simulated insertion forces and the experimental insertion forces decrease as the insertion speeds increase. In the experimental results, the peak insertion forces are decreased by 24% (v=3 mm/s) and 43% (v=7.5 mm/s) compared to v=0.5 mm/s. The small decrease of the simulated insertion forces when speed increases can be directly seen from Table 4. However, in these two figures, there are notable discrepancies between the simulated insertion force and the experimental data.

In FIG. 40B and FIG. 40C, the Stribeck friction model was used. It was found that in the present experiment, the speed related friction force comprised only a small portion of the total sensed insertion force from Eq. 28. The difference between the static and kinetic friction coefficients used was small due to use of lubricant. Therefore, the contribution of the decrease in friction to the total insertion force was comparatively small. One of skill in the art will appreciate that the static and kinetic friction coefficients and the total insertion force will vary predictably in relation to certain adjustments to the experimental model.

When the insertion speed increases in certain embodiments, the pressure distribution of the contacting area between the electrode array and the scala tympani can change. In certain arrangements, this may cause a substantial decrease in the sensed total insertion force. In the present experiment, the inventors have introduced certain adaptations of the model based on considerations of the hydrodynamic effect of the lubricant. When the relative speed between the electrode array and the scala tympani model exceeds an upper limit for the particular parameters considered, the lubricant's hydrodynamic effect helps form a macro-invisible layer of liquid. The layer of liquid contributes to distancing the flexible electrode array away from the external wall of the scala tympani model. Therefore, in the present examples, the pressure distribution may be adjusted and contribute to a more significant decrease in the insertion force than that which is predicted using the Stribeck model.

### Statistical Analysis

Based on the experimental results inventors collected with each insertion speed, inventors derived safety boundaries for the insertion forces. From ten experimental force profiles in each group at any given speed, a log plot logₑ(Fᵢₙₛ) versus θ_{con} was generated. FIG. 41 illustrates the log plot of v=0.5 mm/s and other groups share a similar graph. These plots are illustrative and not limiting, as other insertion speeds are permissible and may be employed by one of skill in the art. The inventors apply a linear regression, Eq. 32, to solve for coefficients c₁ and c₂. The use of regression over logₑ (Fᵢₙₛ) allows a linearization of the non-linear regression problem discussed in various literature.

logₑ(Fᵢₙₛ(θ_{con}))=c₁θ_{con}+c₂ (32)

In accordance with the present model, the exponential of Eq. (32) results in the non-linear fitting model, Eq. (33)

Fᵢₙₛ(θ_{con})=e^{c}^{₁}^{θ}^{_{con}}^{+c}^{₂} (33)

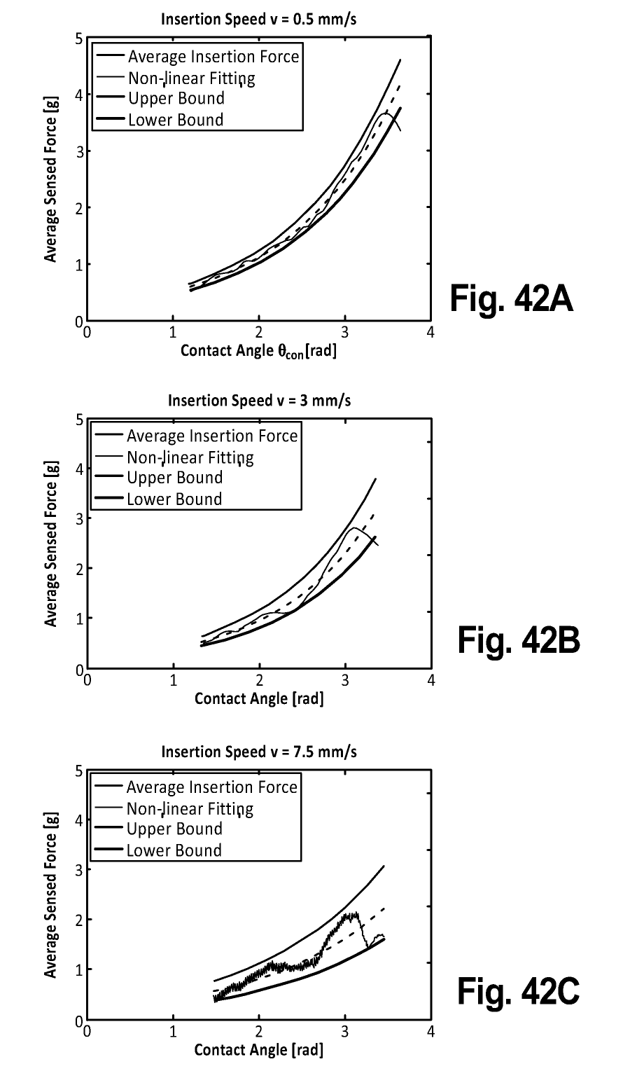

FIGS. 42A, B and C depict the non-linear fitting results for three different insertion speeds, in accordance with the present embodiments. From the fitted curves, it is evident that insertion forces associated with the selected cochlear implant system and technique decrease with an increase in the insertion speed. In the present example, 95% confidence intervals for the insertion force were statistically generated for the fitted model in Eq. (33). These intervals combined with the fitted model are also shown in FIGS. 42A, B and C, as upper and lower boundaries. These upper and lower boundaries are included in part due to their expected usefulness for surgeons. During surgery, surgeons typically will make a judgment to determine the insertion depth at which deeper insertion is likely to cause potential traumas. The present analysis provides surgeons with additional tools by which to improve their case-by-case judgments. In addition, these results may be leveraged for robot-assisted cochlear implant surgery.

In cochlear implant surgery, small insertion forces are important in avoiding trauma throughout the entire insertion process. Preliminary experiments indicate that the insertion force is not only related to the insertion angle (which in turn depends upon insertion depth), but it is also a function of the insertion speed. As noted above, the Stribeck friction model may be employed to correlate the insertion force to the insertion speed. Simulations are found to show that the Stribeck friction model together with a linear contact pressure distribution is an effective tool in explaining the observed behaviors in preliminary experiments. Statistical experiments validated the Stribeck model. Inventors proposed possible analysis on lubricant's hydrodynamic effects that may reduce the contact pressure and hence further decrease the insertion force. As disclosed above, statistical data is used to generate statistical safety boundaries. In one or more embodiments, the statistical safety boundaries are applied to provide predictive information for insertion speed force feedback in robot-assisted cochlear implant surgery. In yet other embodiments, the disclosures herein may be applied to calibrating and validating the Stribeck model on cadaver temporal bones.

### Desired Electrode Shape Determination

While the above discussion focuses on particular embodiments of an electrode array for cochlear implant, modified electrode arrays may also be desirable. In certain experiments, the inventors have found that the electrode array shape may be improved to further reduce insertion forces and reduce the risk of damage during insertion of the cochlear implant.

In certain applications, steerable electrodes for cochlear implant surgery are actuated by an embedded actuation thread that controls the shape of the electrode as it is bent. The problem of finding the optimal radial placement of the actuation wire along the length of the electrode is addressed below. An electrode can be produced that can approximate the shape of the cochlea very closely, throughout the different electrode insertion phases. The discussion below provides a combined modal approach for the direct kinematics modeling of the electrode with an elasticity analysis based on the Chain Algorithm to calibrate a given electrode with a given radial positioning function of the actuation thread. A weighted objective function is defined to characterize the performance of a given electrode for a complete insertion while allowing different weights to address shallow and deep insertions. This objective function may be used to drive the desired shape determination of the electrode in order to find an optimal radial positioning of the actuation wire along the different cross sections of the electrode, according to the selected parameters. The present disclosed technique is shown to be applicable to electrode designs that use different actuation methods.

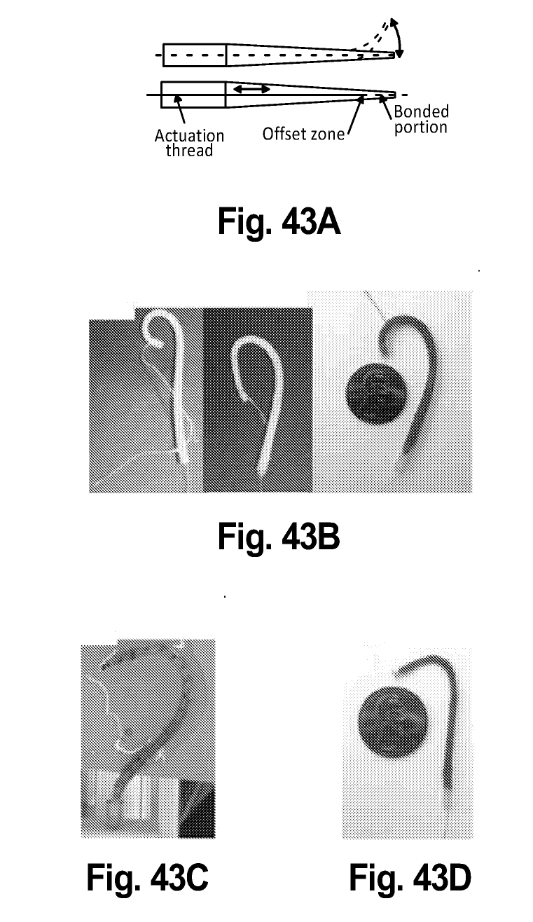

In the preceding sections, inventors disclosed experimental evaluations for steerable cochlear implant electrodes. The steerable electrode shown in FIG. 43 is fabricated from flexible silicone rubber and it is actuated by an embedded actuation thread. In other embodiments, different materials such as may be suitable, as one of skill in the art will appreciate. FIGS. 43A-D depict one embodiment for a steerable electrode with its actuation based on an embedded offset Kevlar thread. Other materials may be used for the thread, such as, for example, NiTi. In FIG. 43A, a schematic depiction of steerable electrode bending with the actuation thread, offset zone and bonded portions are shown. The actuation thread runs along the electrode and it is radially offset from the center of the electrode. The amount of radial offset of the actuation thread determines the shape of the electrode as it bends, as shown in FIG. 43B. FIGS. 43C and D provide alternate views with different bending characteristics with a 3:1 steerable electrode model and a 2:1 steerable electrode model, respectively. Inventors have identified the problem of finding an optimized function of radial offset, as a key means for enhancing the performance of the disclosed steerable electrodes.

The problem of finding the optimal insertion path planning and actuation of a given steerable electrode has been addressed above. Below, an algorithm is disclosed that calculates the desired radial positions (or offset function) of the actuation wire in order to achieve desired insertion characteristics for the electrode. A performance measure is disclosed that quantifies the performance of the steerable electrode as it is inserted into the cochlea, and inventors also present a weighted objective function that quantifies the overall performance of a given electrode throughout the different stages insertion. Inventors present both the experimental and simulation-based calibration of an electrode with a given radial offset of its actuation thread. Inventors also disclose insertion path planning algorithm for electrode insertions based on the experimental/simulation-based calibration methods and a summary overview of the implant desired shape determination method.

### Electrode Modeling and Nomenclature

For clarity, it is assumed that the actuation thread lies in plane. As a result, the electrode bends in the same plane when the actuation thread is pulled. In the extension to a 3D case, each segment of the electrode may be treated as if it bends in a different bending plane as in earlier-discussed embodiments. This assumption is justified, because, among other reasons, the helix of the cochlea has a small and fixed lead angle and the torsion along the curve of the cochlea is small compared to the curvature of the curve (the terms torsion and curvature are used here according to the Ferret-Serret apparatus for describing curves in space).

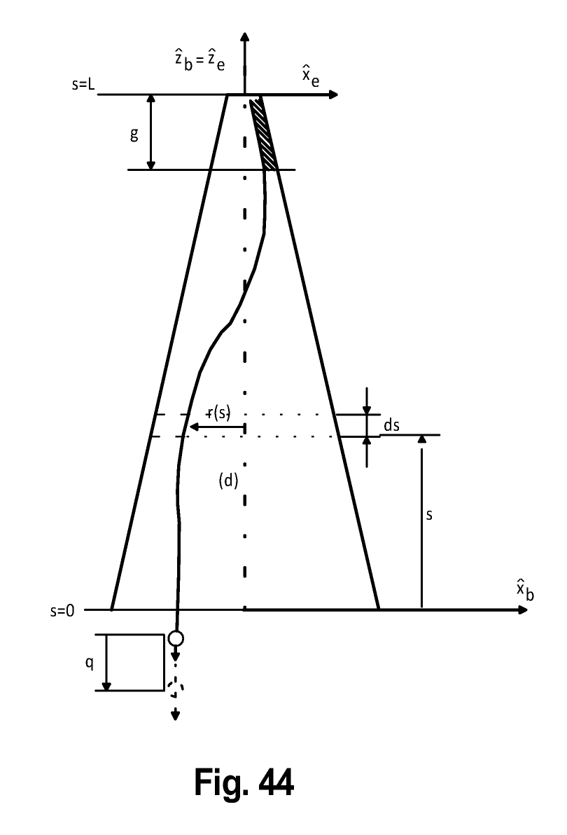

The following nomenclature is used to facilitate the discussion below. The curve of the electrode (or electrode backbone) refers to the axis of the electrode in a bent configuration. {b}={x̂_{b}, ŷ_{b}, ẑ_{b}}describes a coordinate system attached at the base of the electrode, FIG. 44. {e}={x̂ₑ, ŷₑ, ẑₑ} describes a coordinate system attached at the tip of the electrode. {c}={x̂_{c}ŷ_{c}, ẑ_{c}} describes a cochlea-attached coordinate system. This coordinate system is defined such that its x-axis is tangent to the curve of the cochlea at the entrance to the cochlea as it is defined by Cohen's template. The y axis is aligned with the axis of the helix of the cochlea and the z-axis completed this coordinate system to result in a right-handed system. s-arc-length parameter along the axis of the electrode. ε(s)=the axial contraction of the electrode θ(s)=the angle of the vector tangent to the electrode curve as it bends. θ(s) is measured from x̂_{b} to x̂ₑ about ŷₑ according to the right-hand rule. r(s)=the radial position of the actuation thread along the cross section with a coordinate s along the arclength of the electrode backbone. d(s)=the diameter of the electrode along the cross section with a coordinate s along the arc-length of the electrode backbone. q=the actuated joint value (the amount of pull on the actuation thread measured from a configuration in which the electrode is straight). S_{q}=electrode insertion depth. L=overall length of the electrode backbone at a start configuration in which zero force is applied on the electrode and the electrode is straight. d_{b}, dₑ=the diameter of the electrode at its base and at its tip, respectively. Lₐ=the active length of the electrode in which active bending is controllable. Lₐ=L−g, as shown in FIG. 44.

### Performance Measure for Electrode Insertions

An ideal steerable electrode would perfectly match the shape of the cochlea for every insertion depth. This, however, requires an infinite number of actuators. A desirable steerable electrode would very closely match the shape of the cochlea for a broad range of insertion depths. Inventors have found that the size of the electrode limits the feasibility of using more than one actuation thread, in the present embodiments. Since the electrode is a flexible object, it has an infinite number of degrees of freedom, although it uses only one actuation thread. Hence, the steerable electrode may be understood as an underactuated robot whose shape is determined as the one minimizing its elastic and potential energy. The problem of insertion path planning for a given electrode is then defined as finding the actuation value q for every insertion depth S_{q} such that for every insertion depth the electrode approximates the shape of the cochlea to the best of its capacity.

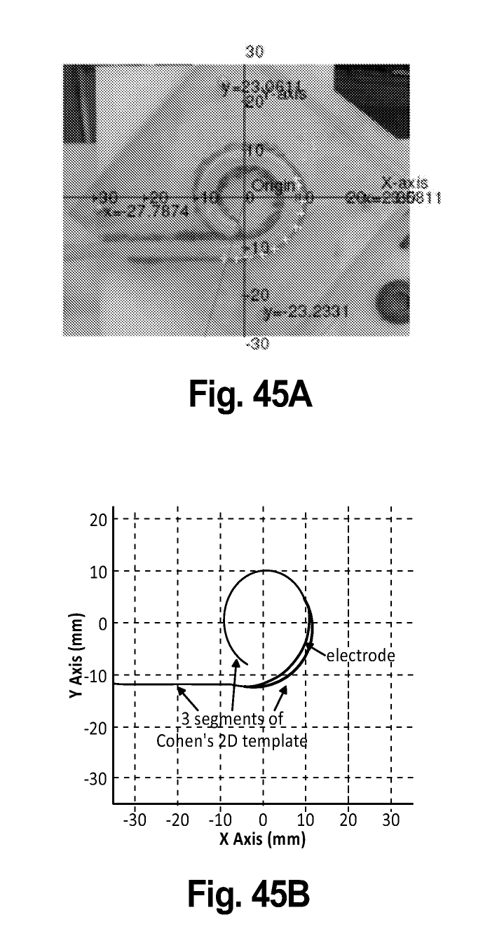

According to the present embodiments, the performance measure that quantifies the quality of an electrode may be defined by an average distance metric E that changes as a function of the insertion depth. The average insertion metric may be calculated according to Eq. (34) where E(θ) is the distance between the inserted portion of the implant and the outer walls of the cochlea and θ is the angle of the electrode curve tangent in x̂_{b}−ẑ_{b} plane. For example this distance metric may be experimentally calculated and shown to be inversely correlated to the insertion forces. FIG. 45A illustrates a digitized image taken during different stages of insertion. FIG. 45B depicts a mathematical model generated from the digitized image. The mathematical model enables the calculation of the distance between the implant and the external walls of the cochlea. For each configuration gauged during the insertion, an average distance was calculated based on the depth or angle of insertion.

$$\overline{E} = {\int }_{{θ}_{min}}^{θ max} E ( θ ) d θ / ( {θ}_{max} - {θ}_{min} ) \qquad ( 34 )$$

The average distance metric as defined in Eq. (34) depends on the insertion depth Sq. A global performance metric E_{g} is defined as the weighted norm of a vector containing all the distance metrics E for all the values of the insertion depths s_{q}ε[0,L]. If the insertion depths are quantized into by an (n+1)−dimensional vector s_{q}=[0,L/n2L/n,3L/n, . . . , L] then it is possible to calculate or experimentally determine the values of the average distance metrics vector e=[Ē(s_{q}=0), . . . , Ē(s_{q}=L)]. The global performance metric is defined by a positive definite quadratic form of Eq. (35). The weight matrix W is a diagonal positive matrix that assigns different penalties for shallow insertions compared to deeper insertions. It has been found that deeper insertions require larger weights, because better approximation of the electrode shape becomes more and more important if one wants to limit the contact angle between the electrode and the outer wall of the cochlea in order to reduce the electrode insertion forces for deep insertions.

E_{g}=e^{t}We (35)

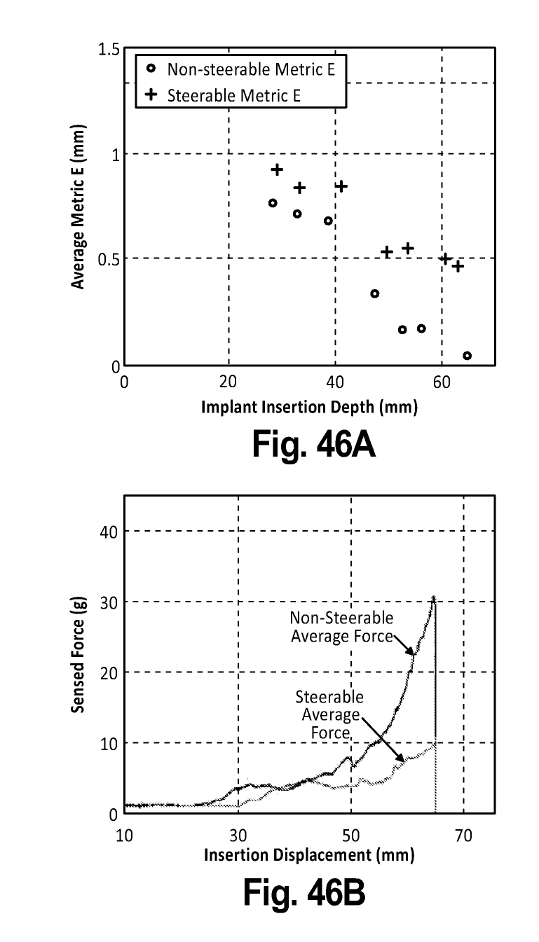

FIG. 46B illustrates electrode insertion forces as a function of insertion displacement for non-steerable and steerable electrode arrays. FIG. 46A illustrates average distance metrics between the implant and the outside wall of the cochlea, as a function of insertion depth. Both non-steerable electrode and steerable electrode metrics are represented. The electrode insertion forces shown in FIG. 46B are inversely correlated to the average distance metrics displayed in FIG. 46A, according to the present embodiments.

### Experimental Calibration and Insertion Path Planning

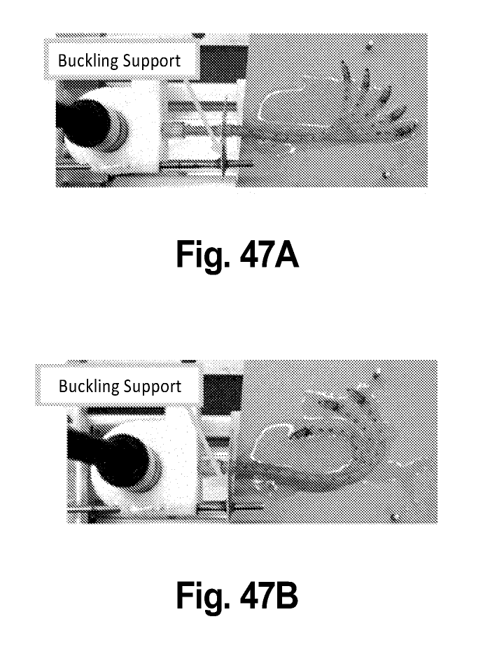

θ(s) may be used to represent the angle of the tangent to the backbone curve of the electrode. s may be used to represent the arc length along the backbone of the electrode where the point s=0 indicates the base and s=L represents the tip of the electrode. Accordingly, q may be used to indicate the value of the joint that controls the bending of the implant. For experimental calibration purposes, the inventors have found it useful to mark R equidistant points along the backbone as shown in FIG. 47. The shape of the electrode can be captured using Z images associated with Z different values of q, in a process similar to that discussed above. For each image one can digitize θ(q)=θ(s=0 . . . L) and then construct a matrix Φε [symbol] that includes the experimental data gathered during the calibration experiment (where Φ_{i,j}=θ(s₁, qⱼ))

According to at least some embodiments, inventors use a modal approach to characterize the shape of the electrode. The shape of the backbone can be described by a modal representation θ(s,q)=Ψ(s)¹a(q),a,Ψε [symbol] where the vectors Ψ(s)=[1,s,s², . . . sⁿ⁻¹]^{t} and a(q) are vectors of modal factors. Accordingly, this vector of modal factors be given by a second series such that a(q)=Aη(q), Aε [symbol] , ηε [symbol] , η(q)=[1,q, q², . . . q^{m-1}]^{t}(36). Using this representation, the problem of calibration using the experimental data matrix Φ can be formulated as given by the algebraic matrix equation in Eq. (36). The numerical values of and Ω and Γ correspond to the R values of s and the Z values of q used to generate the experimental data matrix it. In the present example, the calibration culminates in solving Φ=ΩAΓ for A. The solution for the coefficients matrix A is given by using the matrix kronecker product, Eq. (37).

$$\begin{matrix}Φ = \underbrace{[ \begin{matrix}{Ψ}^{t} ( s = 0 ) \\ ⋮ \\ {Ψ}^{t} ( s = L )\end{matrix} ]}_{Ω} {A}_{n \times m} \underbrace{[ η ( q = 0 ) , … , η ( q = {q}_{max} ) ]}_{Γ} = {Ω}_{r \times n} {A}_{n \times m} {Γ}_{m \times z} & ( 3 ) \\ ⌊ {Γ}^{′} ⊗ Ω ⌋ Vec ( A ) = Vec ( Φ ) & ( 37 )\end{matrix}$$

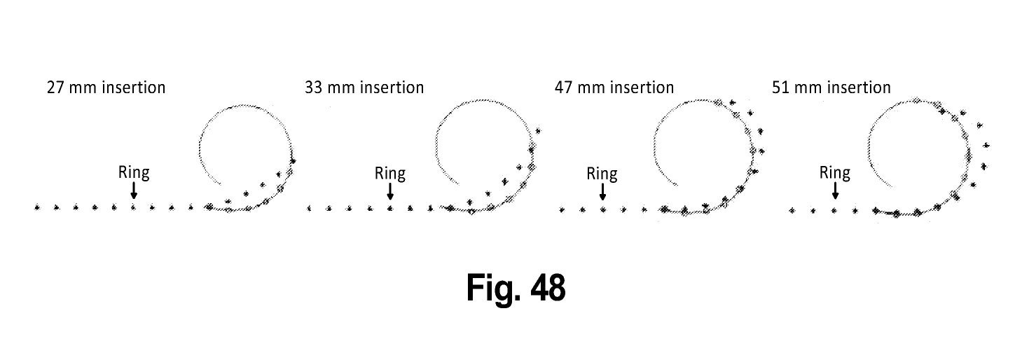

The solution for the desired steering joint value, q, for any range of depth of insertion S_{q} is given by fitting the shape of the electrode to the shape of the cochlea. This is defined by the minimization problem of Eq. (38). S_{q} in Eq. (38) represents the electrode insertion depth and L represents the total length of the electrode. This problem can be solved by using the solution of A and a lookup table and interpolating between its columns or by any other numerical minimization method. Yet other solution methods have been used and can be envisioned by one of skill in the art. Using the experimental calibration model and the path planning algorithm, inventors performed an insertion simulation as shown in FIG. 48. FIG. 48 illustrates an insertion simulation of the electrode based on the calibration of FIG. 47. The steerable implant is marked with (*) and the 2D model of Cohen's template is shown in a solid line for the various configurations and support ring arrangements. The curve of the cochlea model shown in FIG. 48 is includes three segments that correspond to Cohen's template. The shape of the implant is indicated by points.

$$q = {arg min}_{q} {\int }_{L - {s}_{q}}^{L} ( \vert {θ}_{c} ( s ) - θ ( s ) {\vert}^{2} ) d s \qquad ( 38 )$$

### Simulation-Based Calibration of the Electrode and Insertion Path Planning

The calibration algorithm described in the preceding section is based on experimental data gathered from a steerable electrode that is manufactured with a given value of the radial offset r(s) for the actuation thread. Since one objective is to optimize r(s), it is typically undesirable to rely upon an experimental method. It is expected that fabricating many electrodes with different r(s) parameters and characterizing them experimentally is inefficient. The same calibration algorithm described in the previous section may be easily applied to simulation-based calibration. The aforementioned techniques may be adjusted by constructing a static simulation of the electrode and to solve for the shape of the electrode using Finite Element methods or the Chain Algorithm. The details of applying these mathematical methods will be known by one of skill in the art.

### Implant Determination Algorithm

The radial position of the actuation wire can be given as a fraction of its diameter d(s), as in Eq. (39). The diameter of the electrode can be given based on disclosed electrodes and also based on the space available in the scala tympani. In some instances, a typical electrode has a diameter that tapers off at its tip. In yet other instances, atypical tapers may be implemented. Such an electrode can be characterized by Eq. (40) where d_{b} and dₑ are defined as discussed above. The value for β in Eq. (39) may be used to determine the margin between the external walls of the electrode and the closest expected position of the actuation thread to these external walls.

$$\begin{matrix}r ( s ) = d ( s ) \frac{γ ( s )}{2} β where γ ( s ) ∈ [ - 1 , 1 ] and 0 < β < 1 & ( 39 ) \\ d ( s ) = {d}_{b} - ( {d}_{b} - {d}_{e} ) \frac{s}{L} where s ∈ [ 0 , L ] & ( 40 )\end{matrix}$$

The function γ(s) gives the position of the wire within a given cross section of the electrode, Eq. (41).

$$γ ( s ) = - 1 + 2 \tilde{γ} ( s ) ∈ [ - 1 , 1 ] where \tilde{γ} ( s ) = \{ \begin{matrix}\frac{{\sum }_{n = 0}^{m} {{c}_{n} ( \frac{s}{{L}_{a}} )}^{n}}{{\sum }_{n = 0}^{m} {c}_{n}} for s ≦ {L}_{a} \\ \tilde{γ} ( s = {L}_{a} ) for s > {L}_{a}\end{matrix} \} , \tilde{γ} ∈ [ 0 , 1 ] \qquad ( 41 )$$

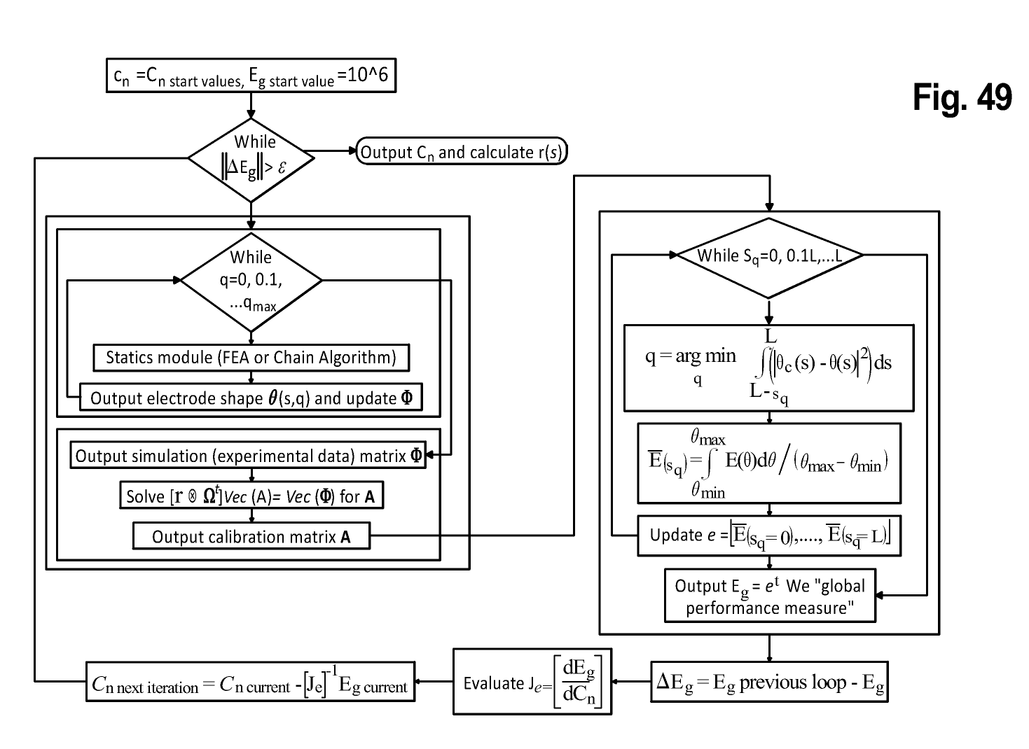

Using this disclosed modal approach, the vector of coefficients Cₙ defines a distinct set of position functions for the actuation thread along the electrode. In the present embodiment, the aim in the desired electrode shape determination algorithm is to calculate this vector of coefficients that minimizes the global performance index E_{g}. FIG. 49 illustrates a block diagram of the implant determination algorithm that provides the optimal radial offset of the actuation wire, according to certain embodiments. The implant determination method applies simulation-based calibration of the electrode and path planning and performance evaluation to optimize the radial offset of the actuation wire. The determination of the desired modal factors Cₙ employs a numerical gradient method for achieving the desired value of the global performance index E_{g}.

The above disclosure provides a method for the determination of certain steerable electrodes for robot-assisted cochlear implant surgery. The method employs an optimal positioning calculation of the actuation thread along the axis of the electrode. Although this method is described with reference to wire-actuated electrodes, it can be extended to other electrode designs using different actuation methods. One of skill in the art can envision such applications. For example, this method can be adapted for calibration and determination of the radial positioning of a stylet in electrodes that use advance-off stylet methods.

The following publications are herein incorporated by reference in their entireties: Zhang, J., Xu, K., Simaan, N., and Manolidis, S., 2006, “A Pilot Study of Robot-Assisted Cochlear Implant Surgery Using Steerable Electrode Arrays,” Medical Image Computing and Computer-Assisted Intervention, 1, pp. 33-40; and U.S. patent application Ser. No. 11/581,899, filed 16 Oct. 2006, entitled “Robot-Assisted Insertion and Monitoring of Passive and Active Bending Cochlear Electrode Arrays,” now US. Patent Publication No. 2007-0225787.

Other embodiments, extensions, and modifications of the ideas presented above are comprehended and are within the reach of one versed in the art upon reviewing the present disclosure. Accordingly, the scope of the present invention in its various aspects are not be limited by the examples presented above. The individual aspects of the present invention, and the entirety of the invention are to be regarded so as to allow for such design modifications and future developments within the scope of the present disclosure. For example, although specific features are described herein in certain combinations, the present invention may be practiced using any combination of any of all or a subset of these features. The present invention is limited only by the claims that follow.

What is claimed is:

1. A system for inserting a steerable array into an anatomical structure of the body, the system comprising:
an insertion module for holding a proximal end of the steerable array;
a force sensor, at the proximal end of the steerable array, configured to detect force on the steerable array and to produce force information;
a position sensor configured to detect a position of the insertion module and to produce position information, the position information including a lateral position along an insertion axis and a first approach angle relative to a first reference axis;
a processor configured to receive the force information from the force sensor and the position information from the position sensor,
wherein the processor outputs performance information to a user, the performance information including an indication of a first differential approach angle relative to an insertion path plan.

2. The system of claim 1, wherein the insertion module is a handheld device that is moved by the user.

3. The system of claim 2, wherein the handheld device provides force feedback to the user based at least in part on an amplification of the detected force on the steerable array.

4. The system of claim 1, wherein the insertion module is adapted to be held and moved by a robotic device.

5. The system of claim 4, wherein the robotic device is controlled by the user.

6. The system of claim 4, wherein the user controls insertion of the bendable array into the anatomical structure along the insertion axis, while the robotic device controls movement of the insertion module in directions other than along the insertion axis based at least in part on the insertion path plan.

7. The system of claim 4, wherein the robotic device provides force feedback to the user based at least in part on an amplification of the detected force on the steerable array.

8. The system of claim 7, wherein the force feedback is provided to the user through a telemanipulation unit that is manipulated by the user to control the robotic device.

9. The system of claim 1, wherein the position information further includes a second approach angle relative to a second reference axis, the second reference axis being orthogonal to the first reference axis.

10. The system of claim 1, wherein the position information further includes a second lateral position along a second axis that is orthogonal to the insertion axis.

11. The system of claim 1, wherein the force sensor is further configured to detect moment on the steerable array and to produce moment information.

12. The system of claim 1, further comprising an orientation sensor configured to detect an orientation of the insertion module to produce orientation information.

13. The system of claim 12, wherein the orientation information includes a roll angle of the insertion module relative to the insertion axis.

14. The system of claim 12, wherein the position sensor and the orientation sensor are implemented as a pose sensor that detects position and orientation on the steerable array.

15. The system of claim 1, wherein the performance information includes an indication of a differential insertion speed relative to the insertion path plan.

16. The system of claim 1, wherein the performance information includes an indication of a differential force on the steerable array relative to the insertion path plan.

17. The system of claim 1, wherein the performance information includes an indication of a differential insertion depth of the steerable array relative to the insertion path plan.

18. The system of claim 1, wherein the performance information includes an indication of safe insertion boundaries of at least one of insertion depth, insertion speed, approach angle, and force on the steerable array.

19. The system of claim 1, wherein the processor outputs a signal to stop insertion of the steerable array if at least one of insertion depth, insertion speed, approach angle, and force on the steerable array are outside of safe insertion boundaries.

20. The system of claim 19, wherein the safe insertion boundaries of at least one of insertion depth, insertion speed, approach angle, and force on the steerable array are based at least in part on a statistical model of the anatomical structure.

21. The system of claim 1, wherein the insertion path plan is based at least in part on a model of the anatomical structure.

22. The system of claim 21, wherein the insertion path plan substantially minimizes expected force between the steerable array and the anatomical structure.

23. The system of claim 1, wherein the insertion path plan is based on a model of the anatomical structure of a patient receiving the steerable array and substantially minimizes expected force between the steerable array and the anatomical structure of the patient.

24. The system of claim 1, wherein the insertion path plan is determined to minimize force arising from contact between the steerable array and the anatomical structure.

25. The system of claim 1, the system further comprising a bending actuator configured to bend an active-bending portion of the steerable array.

26. The system of claim 25, wherein the bending actuator controls the bending of the active-bending portion of the steerable array based at least in part on the insertion path plan.

27. The system of claim 25, wherein the bending actuator controls bending of the active-bending portion of the steerable array by displacing a thread connected to the active-bending portion.

28. The system of claim 27, wherein the thread is connected to the active-bending portion so as to have an offset from a center axis of the steerable array.

29. The system of claim 1, further comprising a display unit for displaying the performance information to the user.

30. The system of claim 29, wherein the display unit indicates a corrective action to the user based at least on the performance information.

31. The system of claim 30, wherein the indicated corrective action includes at least one of an insertion depth correction, an approach angle correction, an insertion speed correction, and a bending actuator displacement correction.

32. The system of claim 1, the insertion module induces vibration in the steerable array to reduce frictional force between the steerable array and the anatomical structure.

33. The system of claim 1, wherein the insertion path plan is determined by a method comprising minimizing a shape difference function, for each of a plurality of insertion depth values, to obtain a value of a bending actuator displacement and a value of the approach angle for each depth value.

34. The system of claim 33, wherein the shape difference function is based at least in part on a shape model of the anatomical structure and a shape model of the steerable array.

35. The system of claim 33, wherein the shape model of the steerable array is experimentally determined.

36. A method for inserting a steerable array into an anatomical structure of the body, the method comprising:
attaching a proximal end of the steerable array to an insertion module;
detecting force on the steerable array, using a force sensor at the proximal end of the steerable array, to produce force information;
detecting a position of the insertion module using a position sensor to produce position information, the position information including a lateral position along an insertion axis and a first approach angle relative to a first reference axis;
receiving the force information from the force sensor and the position information from the position sensor in a processor;
outputting performance information from the processor to a user, the performance information including an indication of a first differential approach angle relative to an insertion path plan.

37. The method of claim 36, wherein the position information further includes a second lateral position along a second axis that is orthogonal to the insertion axis.

38. The method of claim 36, wherein the force sensor further detects moment on the steerable array to produce moment information.

39. The method of claim 36, further comprising detecting an orientation of the steerable array using an orientation sensor to produce orientation information, the orientation information including a roll angle of the insertion module relative to the insertion axis.

40. The method of claim 36, wherein the performance information includes an indication of safe insertion boundaries of at least one of insertion depth, insertion speed, approach angle, and force on the steerable array.

41. The method of claim 36, wherein outputting performance information further comprises outputting a signal to stop insertion of the steerable array if at least one of insertion depth, insertion speed, approach angle, and force on the steerable array are outside of safe insertion boundaries.

42. The method of claim 36, wherein the insertion path plan is determined to minimize force arising from contact between the steerable array and the anatomical structure.

43. The method of claim 36, the insertion module further comprising a bending actuator configured to bend an active-bending portion of the steerable array, the bending actuator controlling the bending of the active-bending portion of the steerable array based at least in part on the insertion path plan.

44. The method of claim 43, wherein the bending actuator controls bending of the active-bending portion of the steerable array by displacing a thread connected to the active-bending portion.

45. The method of claim 36, wherein outputting performance information further comprises indicating to a user a corrective action based at least on the performance information.

46. The method of claim 45, wherein the indicated corrective action includes at least one of an insertion depth correction, an approach angle correction, an insertion speed correction, and a bending actuator displacement correction.

47. The method of claim 36, wherein the insertion path plan is determined by minimizing a shape difference function, for each of a plurality of insertion depth values, to obtain a value of a bending actuator displacement and a value of the approach angle for each depth value.

48. The method of claim 47, wherein the shape difference function is based at least in part on a shape model of the anatomical structure and a shape model of the steerable array.
